(19) United States
(12) Patent Application Publication
(10) Pub. No.: US 2005/0148630 A1
(43) Pub. Date: Jul. 7, 2005

(54) Methods of preventing and treating non-opioid induced gastrointestinal dysfunction

(76) Inventors: Randall L. Carpenter, Waban, MA (US); George E. Dukes, Cary, NC (US); David Jackson, Cape Coral, FL (US); William K. Schmidt, Davis, CA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/998,793

(22) Filed: Nov. 29, 2004

Related U.S. Application Data
(60) Provisional application No. 60/526,327, filed on Dec. 2, 2003.

(51) Int. Cl. A61K 31/445

(52) U.S. Cl. 514/317

(57) ABSTRACT

Methods of preventing and treating non-opioid induced gastrointestinal dysfunction, including chronic constipation, slow colonic transit, low stool frequency, and poor stool consistency, with reduced undesirable side effects are disclosed.

### CROSS REFERENCE TO RELATED APPLICATIONS

This application claims priority to U.S. Application No. 60/526,327 filed Dec. 2, 2003, the entire disclosure of which is incorporated herein by reference.

### FIELD OF THE INVENTION

The present invention generally relates to methods of preventing and treating gastrointestinal dysfunction. More specifically, the present invention relates to methods of preventing and treating non-opioid induced gastrointestinal dysfunction, including chronic constipation, slow colonic transit, low stool frequency, and poor stool consistency.

### BACKGROUND OF THE INVENTION

Constipation is a common symptom that may be idiopathic or due to various identifiable disease processes. Constipation may be defined as a low defecation rate of about three or fewer bowel movements in a 7-day period. Individuals may suffer from a single bout of constipation (acute or current) or the condition may be chronic (recurring and/or of long duration). There are a number of known treatments for constipation, depending upon the severity, duration and cause of the condition. For example, laxatives, fiber, stool softeners, bowel stimulants, and the like have been used to treat constipation. Laxatives are agents that add bulk to intestinal contents, that retain water within the bowel lumen by virtue of osmotic effects, or that stimulate intestinal secretion or motility, thereby increasing the frequency and ease of defecation. Drugs that improve constipation by stimulating gastrointestinal motility by direct actions on the enteric nervous system are under development. Other modalities used to treat constipation include biofeedback and surgery. See, for example, Schiller L. R., Review Article: The Therapy of Constipation,” Aliment. Pharmacol. Ther., 2001, 15(6): 749-63.

It has been suggested that certain opioid antagonists may be used to treat opioid-induced constipation, an undesirable side effect often associated with administration of opiate analgesics, particularly post-operatively or post-partum. See, for example, U.S. Pat. No. 4,987,136 for centrally-acting opioid antagonists. See, for example, U.S. Pat. No. 5,250,542 and U.S. Pat. No. 5,434,171 for peripherally-acting opioid antagonists.

Chronic constipation, also known as functional or idiopathic constipation, may be defined as constipation with no known cause or etiology. U.S. Pat. No. 5,250,542 and U.S. Pat. No. 5,434,171 not only disclose the use of 4-aryl piperidine derivatives at a level of 1 to 500 mg for the treatment of opioid-induced constipation but also idiopathic constipation. Unfortunately, treatment of constipation with 4-aryl piperidine derivatives at lower levels within this disclosed range, including at levels of greater than about 18 mg/day to about 54 mg/day caused undesirable side effects, including abdominal pain, diarrhea, and/or flatulence.

Therefore, it would be desirable to provide methods for preventing and/or treating non-opioid induced chronic constipation with reduced side effects. The methods of the present invention are directed toward these, as well as other, important ends.

### SUMMARY OF THE INVENTION

The methods of the present invention are directed to treating and preventing non-opioid induced gastrointestinal dysfunction, including chronic constipation, slow colonic transit, low stool frequency, and poor stool consistency, with reduced undesirable side effects, including abdominal pain, diarrhea, flatulence, or combinations thereof.

Accordingly, the present invention is directed, in part, to methods of treating or preventing non-opioid induced gastrointestinal dysfunction, comprising the step of:
administering to a patient in need thereof about 0.5 mg/day to about 18 mg/day of at least one 4-aryl-piperidine derivative or a stereoisomer, a prodrug, a pharmaceutically acceptable salt, a hydrate, a solvate, an acid salt hydrate, an N-oxide or an isomorphic crystalline form thereof;
wherein said patient is not receiving chronic or periodic exogenous opioids;
wherein said gastrointestinal dysfunction is chronic constipation, slow colonic transit, low stool frequency, poor stool consistency, or combinations thereof; and
wherein said 4-aryl-piperidine derivative is a compound of formula (IA): 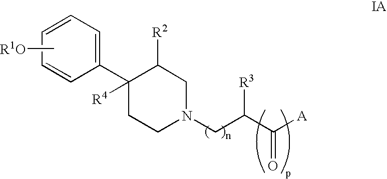
   wherein:
R¹ is hydrogen or alkyl;
R² is hydrogen, alkyl or alkenyl;
R³ is hydrogen, alkyl, alkenyl, aryl, cycloalkyl, cycloalkenyl, cycloalkyl-substituted alkyl, cycloalkenyl-substituted alkyl or aralkyl;
R⁴ is hydrogen, alkyl or alkenyl;
A is OR⁵ or NR⁶R⁷;
R⁵ is hydrogen, alkyl, alkenyl, cycloalkyl, cycloalkenyl, cycloalkyl-substituted alkyl, cycloalkenyl-substituted alkyl, or aralkyl;
R⁶ is hydrogen or alkyl;
R⁷ is hydrogen, alkyl, alkenyl, cycloalkyl, aryl, cycloalkyl-substituted alkyl, cycloalkenyl, cycloalkenyl-substituted alkyl, aralkyl, B, or alkylene substituted B or, together with the nitrogen atom to which they are attached, R⁶ and R⁷ form a heterocyclic ring;
B is 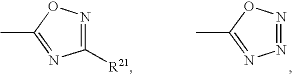
C(═O)W or NR⁸R⁹;
R⁸ is hydrogen or alkyl;
R⁹ is hydrogen, alkyl, alkenyl, cycloalkyl-substituted alkyl, cycloalkyl, cycloalkenyl, cycloalkenyl-substituted alkyl, aryl or aralkyl or, together with the nitrogen atom to which they are attached, R⁸ and R⁹ form a heterocyclic ring;
W is OR¹⁰, NR¹¹R¹², or OE;
R¹⁰ is hydrogen, alkyl, alkenyl, cycloalkyl, cycloalkenyl, cycloalkyl-substituted alkyl, cycloalkenyl-substituted alkyl, or aralkyl;
R¹¹ is hydrogen or alkyl;
R¹² is hydrogen, alkyl, alkenyl, aryl, cycloalkyl, cycloalkenyl, cycloalkyl-substituted alkyl, cycloalkenyl-substituted alkyl, aralkyl or alkylene substituted C(═O)Y or, together with the nitrogen atom to which they are attached, R¹¹ and R¹² form a heterocyclic ring;
E is 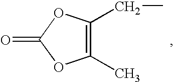
alkylene substituted (C═O)D, or —R¹³OC(═O)R¹⁴;
R¹³ is alkyl substituted alkylene;
R¹⁴ is alkyl;
D is OR⁵ or NR¹⁶R¹⁷;
R¹⁵ is hydrogen, alkyl, alkenyl, cycloalkyl, cycloalkenyl, cycloalkyl-substituted alkyl, cycloalkenyl-substituted alkyl, or aralkyl;
R¹⁶ is hydrogen, alkyl, alkenyl, aryl, aralkyl, cycloalkyl, cycloalkenyl, cycloalkyl-substituted alkyl or cycloalkenyl-substituted alkyl;
R¹⁷ is hydrogen or alkyl or, together with the nitrogen atom to which they are attached, R¹⁶ and R¹⁷ form a heterocyclic ring;
Y is OR¹⁸ or NR¹⁹R²⁰;
R¹⁸ is hydrogen, alkyl, alkenyl, cycloalkyl, cycloalkenyl, cycloalkyl-substituted alkyl, cycloalkenyl-substituted alkyl, or aralkyl;
R¹⁹ is hydrogen or alkyl;
R²⁰ is hydrogen, alkyl, alkenyl, aryl, cycloalkyl, cycloalkenyl, cycloalkyl-substituted alkyl, cycloalkenyl-substituted alkyl, or aralkyl or, together with the nitrogen atom to which they are attached, R¹⁹ and R²⁰ form a heterocyclic ring;
R²¹ is hydrogen or alkyl;
n is 0 to 4;
p is 0 or 1; and
provided that R¹⁰ is not hydrogen, when R¹ is hydrogen, R² is methyl, R³ is cycloalkyl-substituted alkyl, and R⁴ is methyl; and
provided that R¹⁰ is not alkyl, when R¹ is hydrogen, R² is methyl, R³is aralkyl, and R⁴ is methyl.

In addition, the present invention is directed, in part, to methods of treating or preventing non-opioid induced gastrointestinal dysfunction, comprising the step of:
administering to a patient in need thereof about 0.5 mg/day to about 18 mg/day of at least one 4-aryl-piperidine derivative or a stereoisomer, a prodrug, a pharmaceutically acceptable salt, a hydrate, a solvate, an acid salt hydrate, an N-oxide or an isomorphic crystalline form thereof;
wherein said patient is not receiving chronic or periodic exogenous opioids;
wherein said gastrointestinal dysfunction is chronic constipation, slow colonic transit, low stool frequency, poor stool consistency, or combinations thereof;
wherein said 4-aryl-piperidine derivative is [[2-[[-4-(3-hydroxyphenyl)-3,4-dimethylpiperidin-1-yl]methyl]-3-phenylpropanoyl]amino]acetic acid. Preferably, the [[2-[[-4-(3-hydroxyphenyl)-3,4-dimethylpiperidin-1-yl]methyl]-3-phenylpropanoyl]amino]acetic acid is in hydrate form, more preferably, [[2-[[-4-(3-hydroxyphenyl)-3,4-dimethylpiperidin-1-yl]methyl]-3-phenylpropanoyl]amino]acetic acid dihydrate, even more preferably in substantially pure isomeric form, most especially [[(2S)-2-[[(3R,4R)-4-(3-hydroxyphenyl)-3,4-dimethylpiperidin-1-yl]methyl]-3-phenylpropanoyl]amino]acetic acid dihydrate (alvimopan).

In certain preferred embodiments of the invention, said 4-aryl-piperidine derivative or stereoisomer, prodrug, pharmaceutically acceptable salt, hydrate, solvate, acid salt hydrate, N-oxide or isomorphic crystalline form thereof is administered at a level of at least about 0.75 mg/day, more preferably at a level of at least about 1 mg/day, even more preferably at a level of at least about 2 mg/day, and yet even more preferably at a level of at least about 3 mg/day.

In certain preferred embodiments of the invention, said 4-aryl-piperidine derivative or stereoisomer, prodrug, pharmaceutically acceptable salt, hydrate, solvate, acid salt hydrate, N-oxide or isomorphic crystalline form thereof is administered at a level of less than about 15 mg/day, more preferably at a level of less than about 12 mg/day, even more preferably at a level of less than about 9 mg/day, and yet even more preferably at a level of less than about 6 mg/day.

In certain preferred embodiments of the invention, said 4-aryl-piperidine derivative or stereoisomer, prodrug, pharmaceutically acceptable salt, hydrate, solvate, acid salt hydrate, N-oxide or isomorphic crystalline form thereof is administered for at least about 1 day, more preferably for at least about 2 days, even more preferably for at least about 3 days, yet even more preferably, for at least about 5 days, and further more preferably, for at least about 7 days.

### BRIEF DESCRIPTION OF THE DRAWINGS

FIG. 1 shows bowel movement frequency: bowel movement (BM) or spontaneous complete bowel movement (SCBM) per week (as a change from baseline). Alvimopan treatment increased both the mean total bowel movement increase from baseline and the mean SCBM change from baseline.

FIG. 2 shows stool consistency (with Bristol Stool Form Scale). Alvimopan treatment increased the mean bowel movement Bristol Stool Form Scale score indicating a change to a softer stool.

FIG. 3 shows bowel movement characteristics as percent of bowel movements, no/mild straining or discomfort and improved bowel movement satisfaction. Alvimopan treatment increased the % of bowel movements with no/mild straining or discomfort and improved satisfaction with bowel movements.

### DETAILED DESCRIPTION OF THE INVENTION

The methods of the present invention are directed to treating and preventing non-opioid induced gastrointestinal dysfunction, including chronic constipation, slow colonic transit, low stool frequency, and poor stool consistency, with reduced undesirable side effects, including abdominal pain, diarrhea, flatulence or combinations thereof, comprising the step of:
administering to a patient in need thereof of a low dose of a 4-aryl-piperidine derivative or a stereoisomer, a prodrug, a pharmaceutically acceptable salt, a hydrate, a solvate, an acid salt hydrate, an N-oxide or an isomorphic crystalline form thereof;
wherein said patient is not receiving chronic or periodic exogenous opioids.

As employed above and throughout the disclosure, the following terms, unless otherwise indicated, shall be understood to have the following meanings.

As used herein, “alkyl” refers to an optionally substituted, saturated straight, branched, or cyclic hydrocarbon having from about 1 to about 20 carbon atoms (and all combinations and subcombinations of ranges and specific numbers of carbon atoms therein), with from about 1 to about 8 carbon atoms, herein referred to as “lower alkyl”, being preferred. “Branched” refers to an alkyl group in which a lower alkyl group, such as methyl, ethyl, or propyl, is attached to a linear alkyl chain. In certain preferred embodiments, the alkyl group is a C₁-C₅ alkyl group, i.e., a branched or linear alkyl group having from 1 to about 5 carbons. In other preferred embodiments, the alkyl group is a C₁-C₃ alkyl group, i.e., a branched or linear alkyl group having from 1 to about 3 carbons. Exemplary alkyl groups include methyl, ethyl, n-propyl, isopropyl, butyl, isobutyl, sec-butyl, tert-butyl, pentyl, hexyl, heptyl, octyl, nonyl, and decyl. “Lower alkyl” refers to an alkyl group having 1 to about 6 carbon atoms. Preferred alkyl groups include the lower alkyl groups of 1 to about 3 carbons. Alkyl groups include, but are not limited to, methyl, ethyl, n-propyl, isopropyl, n-butyl, isobutyl, t-butyl, n-pentyl, cyclopentyl, isopentyl, neopentyl, n-hexyl, isohexyl, cyclohexyl, cyclooctyl, adamantyl, 3-methylpentyl, 2,2-dimethylbutyl, and 2,3-dimethylbutyl.

As used herein, “alkylene” refers to a bivalent alkyl radical having the general formula —(CH₂)ₙ—, where n is 1 to 10, and all combinations and subcombinations of ranges therein. The alkylene group may be straight, branched or cyclic. Non-limiting examples include methylene, methylene (—CH₂—), ethylene (—CH₂CH₂—), propylene (—(CH₂)₃—), trimethylene, pentamethylene, and hexamethylene. There may be optionally inserted along the alkylene group one or more oxygen, sulfur or optionally substituted nitrogen atoms, wherein the nitrogen substituent is alkyl as described previously. Alkylene groups can be optionally substituted. The term “lower alkylene” herein refers to those alkylene groups having from about 1 to about 6 carbon atoms. Preferred alkylene groups have from about 1 to about 4 carbons.

As used herein, “aralkylene” refers to a bivalent alkyl radical having the general formula —(CH₂)ₙ—, wherein any one of the hydrogens on the alkylene radical is replaced by an aryl group, and where n is 1 to 10. Aralkylene groups can be optionally substituted. Non-limiting examples include phenylmethylene, 2-phenyltrimethylene, 3-(p-anisyl)-pentamethylene, and 2-(m-trifluromethylphenyl)-hexamethylene. Aralkylene groups can be substituted or unsubstituted. The term “lower aralkylene” herein refers to those aralkylene groups having from about 1 to about 6 carbon atoms in the alkylene portion of the aralkylene group.

As used herein, “alkenyl” refers to a monovalent alkyl radical containing at least one carbon-carbon double bond and having from 2 to about 10 carbon atoms in the chain, and all combinations and subcombinations of ranges therein. Alkenyl groups can be optionally substituted. In certain preferred embodiments, the alkenyl group is a C₂-C₁₀ alkyl group, i.e., a branched or linear alkenyl group having from 2 to about 10 carbons. In other preferred embodiments, the alkenyl group is a C₂-C₆ alkenyl group, i.e., a branched or linear alkenyl group having from 2 to about 6 carbons. In still other preferred embodiments, the alkenyl group is a C₃-C₁₀ alkenyl group, i.e., a branched or linear alkenyl group having from about 3 to about 10 carbons. In yet other preferred embodiments, the alkenyl group is a C₂-C₅ alkenyl group, i.e., a branched or linear alkenyl group having from 2 to about 5 carbons. Exemplary alkenyl groups include, for example, vinyl, propenyl, butenyl, pentenyl hexenyl, heptenyl, octenyl, nonenyl and decenyl groups.

As used herein, the term “alkenylene” refers to an alkylene group containing at least one carbon-carbon double bond. Exemplary alkenylene groups include, for example, ethenylene (—CH═CH—) and propenylene (—CH═CHCH₂—). Preferred alkenylene groups have from 2 to about 4 carbons.

As used herein, “aryl” refers to an optionally substituted, mono-, di-, tri-, or other multicyclic aromatic ring system having from about 5 to about 50 carbon atoms (and all combinations and subcombinations of ranges and specific numbers of carbon atoms therein), with from about 6 to about 10 carbons being preferred. Non-limiting examples include, for example, phenyl, naphthyl, anthracenyl, and phenanthrenyl.

As used herein, “aralkyl” refers to alkyl radicals bearing an aryl substituent and have from about 6 to about 50 carbon atoms (and all combinations and subcombinations of ranges and specific numbers of carbon atoms therein), with from about 6 to about 10 carbon atoms being preferred. Aralkyl groups can be optionally substituted in either the aryl or alkyl portions. Non-limiting examples include, for example, phenylmethyl (benzyl), diphenylmethyl, triphenylmethyl, phenylethyl, diphenylethyl and 3-(4-methylphenyl)propyl.

As used herein, “heteroaryl” refers to an optionally substituted, mono-, di-, tri-, or other multicyclic aromatic ring system that includes at least one, and preferably from 1 to about 4 sulfur, oxygen, or nitrogen heteroatom ring members. Heteroaryl groups can have, for example, from about 3 to about 50 carbon atoms (and all combinations and subcombinations of ranges and specific numbers of carbon atoms therein), with from about 4 to about 10 carbons being preferred. Non-limiting examples of heteroaryl groups include, for example, pyrryl, furyl, pyridyl, 1,2,4-thiadiazolyl, pyrimidyl, thienyl, isothiazolyl, imidazolyl, tetrazolyl, pyrazinyl, pyrimidyl, quinolyl, isoquinolyl, thiophenyl, benzothienyl, isobenzofuryl, pyrazolyl, indolyl, purinyl, carbazolyl, benzimidazolyl, and isoxazolyl.

As used herein, “cycloalkyl” refers to an optionally substituted, alkyl group having one or more rings in their structures having from about 3 to about 20 carbon atoms (and all combinations and subcombinations of ranges and specific numbers of carbon atoms therein), with from about 3 to about 10 carbon atoms being preferred, with from about 3 to about 8 carbon atoms being more preferred, with from about 3 to about 6 carbon atoms being even more preferred. Multi-ring structures may be bridged or fused ring structures. The cycloalkyl group may be optionally substituted with, for example, alkyl, preferably C₁-C₃ alkyl, alkoxy, preferably C₁-C₃ alkoxy, or halo. Non-limiting examples include, for example, cyclopropyl, cyclobutyl, cyclopentyl, cyclohexyl, cycloheptyl cyclooctyl, and adamantyl.

As used herein, “cycloalkyl-substituted alkyl” refers to a linear alkyl group, preferably a lower alkyl group, substituted at a terminal carbon with a cycloalkyl group, preferably a C₃-C₈ cycloalkyl group. Non-limiting examples include, for example, cyclohexylmethyl, cyclohexylethyl, cyclopentylethyl, cyclopentylpropyl, cyclopropylmethyl and the like.

As used herein, “cycloalkenyl” refers to an olefinically unsaturated cycloalkyl group having from about 4 to about 10 carbons, and all combinations and subcombinations of ranges therein. In preferred embodiments, the cycloalkenyl group is a C₅-C₈ cycloalkenyl group, i.e., a cycloalkenyl group having from about 5 to about 8 carbons.

As used herein, “alkylcycloalkyl” refers to an optionally substituted ring system comprising a cycloalkyl group having one or more alkyl substituents. Non-limiting examples include, for example, alkylcycloalkyl groups include 2-methylcyclohexyl, 3,3-dimethylcyclopentyl, trans-2,3-dimethylcyclooctyl, and 4-methyldecahydronaphthalenyl.

As used herein, “heteroaralkyl” refers to an optionally substituted, heteroaryl substituted alkyl radicals having from about 2 to about 50 carbon atoms (and all combinations and subcombinations of ranges and specific numbers of carbon atoms therein), with from about 6 to about 25 carbon atoms being preferred. Non-limiting examples include 2-(1H-pyrrol-3-yl)ethyl, 3-pyridylmethyl, 5-(2H-tetrazolyl)methyl, and 3-(pyrimidin-2-yl)-2-methylcyclopentanyl.

As used herein, “heterocycloalkyl” refers to an optionally substituted, mono-, di-, tri-, or other multicyclic aliphatic ring system that includes at least one, and preferably from 1 to about 4 sulfur, oxygen, or nitrogen heteroatom ring members. Heterocycloalkyl groups can have from about 3 to about 20 carbon atoms (and all combinations and subcombinations of ranges and specific numbers of carbon atoms therein), with from about 4 to about 10 carbons being preferred. The heterocycloalkyl group may be unsaturated, and may also be fused to aromatic rings. Non-limiting examples include, for example, tetrahydrofuranyl, tetrahydrothienyl, piperidinyl, pyrrolidinyl, isoxazolidinyl, isothiazolidinyl, pyrazolidinyl, oxazolidinyl, thiazolidinyl, piperazinyl, morpholinyl, piperadinyl, decahydroquinolyl, octahydrochromenyl, octahydro-cyclopenta[c]pyranyl, 1,2,3,4,-tetrahydroquinolyl, octahydro-[2]pyrindinyl, decahydro-cycloocta[c]furanyl, and imidazolidinyl.

As used herein, the term “spiroalkyl” refers to an optionally substituted, alkylene diradical, both ends of which are bonded to the same carbon atom of the parent group to form a spirocyclic group. The spiroalkyl group, taken together with its parent group, as herein defined, has 3 to 20 ring atoms. Preferably, it has 3 to 10 ring atoms. Non-limiting examples of a spiroalkyl group taken together with its parent group include 1-(1-methyl-cyclopropyl)-propan-2-one, 2-(1-phenoxy-cyclopropyl)-ethylamine, and 1-methyl-spiro[4.7]dodecane.

As used herein, the term “alkoxy” refers to an optionally substituted alkyl-O— group wherein alkyl is as previously defined. Non-limiting examples include, for example, include methoxy, ethoxy, n-propoxy, i-propoxy, n-butoxy, and heptoxy.

As used herein, the term “aryloxy” refers to an optionally substituted aryl-O— group wherein aryl is as previously defined. Non-limiting examples include, for example, phenoxy and naphthoxy.

As used herein, the term “aralkoxy” refers to an optionally substituted aralkyl-O— group wherein aralkyl is as previously defined. Non-limiting examples include, for example, benzyloxy, 1-phenylethoxy, 2-phenylethoxy, and 3-naphthylheptoxy.

As used herein, the term “aryloxyaryl” refers to an aryl group with an aryloxy substituent wherein aryloxy and aryl are as previously defined. Aryloxyaryl groups can be optionally substituted. Non-limiting examples include, for example, phenoxyphenyl, and naphthoxyphenyl.

As used herein, the term “heteroarylaryl” refers to an aryl group with a heteroaryl substituent wherein heteroaryl and aryl are as previously defined. Heteroarylaryl groups can be optionally substituted. Non-limiting examples include, for example, 3-pyridylphenyl, 2-quinolylnaphthalenyl, and 2-pyrrolylphenyl.

As used herein, the term “alkoxyaryl” refers to an aryl group bearing an alkoxy substituent wherein alkoxy and aryl are as previously defined. Alkoxyaryl groups can be optionally substituted. Non-limiting examples include, for example, para-anisyl, meta-t-butoxyphenyl, and methylendioxyphenyl.

As used herein, the term “carbon chain of said alkoxy interrupted by a nitrogen atom” refers to a carbon chain of an alkoxy group, wherein a nitrogen atom has been inserted between two adjacent carbon atoms of the carbon chain and wherein alkoxy is as previously defined. Both the alkoxy group and the nitrogen atom can be optionally substituted. Exemplary groups include —OCH₂CH₂N(CH₃)CH₂CH₃ and —OCH₂CH₂NHCH₃.

As used herein, the term “heterocycloalkylheteroaryl” refers to an heteroaryl group with a heterocycloalkyl substituent wherein heterocycloalkyl and heteroaryl are as previously defined. Heterocycloalkylheteroaryl groups can be optionally substituted. Exemplary heterocycloalkylheteroaryl groups include 3-[N-morpholinyl]pyridine and 3-[2-piperidinyl]pyridine.

As used herein, the term “heteroarylheteroaryl” refers to a heteroaryl group with a heteroaryl substituent wherein heteroaryl is as previously defined. Heteroarylherteroaryl groups can be optionally substituted. Exemplary heteroarylheteroaryl groups include 4-[3-pyridyl]pyridine and 2-[2-quinolyl]quinuclidine.

As used herein, the term “aralkoxyaryl” refers to an aryl group with an aralkoxy substituent wherein aralkoxy and aryl are as previously defined. Aralkoxyaryl groups can be optionally substituted. Exemplary aralkoxyaryl groups include benzyloxyphenyl and meta-toluenyloxyphenyl.

As used herein, the term “arylheteroaryl” refers to a heteroaryl group with an aryl substituent wherein aryl and heteroaryl are as previously defined. Arylheteroaryl groups can be optionally substituted. Exemplary arylheteroaryl groups include 3-phenylpyridyl and 2-naphthalenylquinolinyl.

As used herein, the term “alkoxyheteroaryl” refers to an heteroaryl group with an alkoxy substituent wherein alkoxy and heteroaryl are as previously defined. Alkoxyheteroaryl groups can be optionally substituted. Exemplary alkoxyheteroaryl groups include 2-methoxypyridine and 6-n-propoxyquinoline.

As used herein, “bicycloalkyl” refers to an optionally substituted, alkyl group having two bridged rings in its structure and having from about 7 to about 20 carbon atoms (and all combinations and subcombinations of ranges and specific numbers of carbon atoms therein), with from about 7 to about 15 carbon atoms being preferred. Exemplary bicycloalkyl-ring structures include, but are not limited to, norbornyl, bornyl, [2.2.2]-bicyclooctyl, cis-pinanyl, trans-pinanyl, camphanyl, iso-bornyl, and fenchyl.

As used herein, “bicycloalkenyl” refers to an optionally substituted, alkenyl group having two bridged rings in its structure and having from about 7 to about 20 carbon atoms (and all combinations and subcombinations of ranges and specific numbers of carbon atoms therein), with from about 7 to about 15 carbon atoms being preferred. Exemplary bicycloalkenyl-ring structures include, but are not limited to, bicyclo[2.2.1]hept-5-en-2-yl, bornenyl, [2.2.2]-bicyclooct-5-en-2-yl, α-pinenyl, β-pinenyl, camphenyl, and fenchyl.

As used herein, “carboxy” refers to a —C(═O)OH group.

As used herein, “alkanoyl” refers to a —C(═O)-alkyl group, wherein alkyl is as previously defined. Exemplary alkanoyl groups include acetyl (ethanoyl), n-propanoyl, n-butanoyl, 2-methylpropanoyl, n-pentanoyl, 2-methylbutanoyl, 3-methylbutanoyl, 2,2-dimethylpropanoyl, heptanoyl, decanoyl, and palmitoyl.

As used herein, “alkoxy-alkyl” refers to an alkyl-O-alkyl group where alkyl is as previously described.

As used herein, “heterocyclic” refers to a monocyclic or multicyclic ring system carbocyclic radical containing from about 4 to about 10 members, and all combinations and subcombinations of ranges therein, wherein one or more of the members is an element other than carbon, for example, nitrogen, oxygen or sulfur. The heterocyclic group may be aromatic or nonaromatic. Non-limiting examples include, for example, pyrrole and piperidine groups.

As used herein, “halo” refers to fluoro, chloro or bromo.

Typically, substituted chemical moieties include one or more substituents that replace hydrogen. Exemplary substituents include, for example, halo (e.g., F, Cl, Br, I), alkyl, cycloalkyl, alkylcycloalkyl, alkenyl, alkynyl, aralkyl, aryl, heteroaryl, heteroaralkyl, spiroalkyl, heterocycloalkyl, hydroxyl (—OH), nitro (—NO₂), cyano (—CN), amino (—NH₂), —N-substituted amino (—NHR″), —N,N-disubstituted amino (—N(R″)R″), carboxyl (—COOH), —C(═O)R″, —OR″, —C(═O)OR″, —NHC(═O)R″, aminocarbonyl (—C(═O)NH₂), —N-substituted aminocarbonyl (—C(═O)NHR″), —N,N-disubstituted aminocarbonyl (—C(═O)N(R″)R″), thiol, thiolato (SR″), sulfonic acid (SO₃H), phosphonic acid (PO₃H), S(═O)₂R″, S(═O)₂NH₂, S(═O)₂ NHR″, S(═O)₂NR″R″, NHS(═O)₂R″, NR″S(═O)₂R″, CF₃, CF₂CF₃, NHC(═O)NHR″, NHC(═O)NR″R″, NR″C(═O)NHR″, NR″C(═O)NR″R″, NR″C(═O)R″ and the like. In relation to the aforementioned substituents, each moiety R″ can be, independently, any of H, alkyl, cycloalkyl, alkenyl, aryl, aralkyl, heteroaryl, or heterocycloalkyl, for example.

As used herein, the term “gastrointestinal dysfunction” refers to a collection of conditions and symptoms including chronic constipation, slow colonic transit, low stool frequency, poor stool consistency, and combinations thereof.

As used herein, the term “chronic constipation” refers to a low defecation rate of about three or fewer bowel movements in about a 7-day period with no known cause or etiology.

As used herein, the term “slow colonic transit” refers to hypomotility of the colon or colonic inertia as defined by standard radiographic evaluations (e.g., radio-opaque markers or colonic transit scintigraphy).

As used herein, the term “oral-cecal transit time” refers to the time required for a standard marker (e.g., lactulose in the lactulose hydrogen breath test) to pass from the mouth to the cecum. As used herein, the phrase “does not substantially affect oral-cecal transit time” refers to no more than about a 10% change in the time required for a standard marker to pass from the mouth to the cecum.

As used herein, the term “low stool frequency” refers to a defecation rate of about three or fewer bowel movements in about a 7-day period.

As used herein, the term “poor stool consistency” refers to hard, lumpy stools (Bristol Stool Form Scale type 1 or 2; Heaton, et al., Gut, 1991, 73-79).

As used herein, the term “non-opioid induced” refers to a condition, in the case of this present invention, gastrointestinal dysfunction, that is not primarily caused by the administration of one or more exogenous opioids.

As used herein, term “irritable bowel syndrome” refers to a gastrointestinal disease, as defined in Isselbacher, et al. (editors), Harrison's Principles of Internal Medicine, 13^{th} Edition, New York: McGraw-Hill, Inc., 1994, 1421-1422, the disclosure of which is incorporated herein by reference in its entirety.

As used herein, the phrase “patient is not receiving chronic or periodic exogenous opioids” refers to a patient who is administered chronically or periodically less than about 0.5 ml oral opium tincture, or about 5 ml oral paregoric, or equivalent antidiarrheal doses of morphine, codeine, loperamide, diphenoxylate, or other natural or synthetic opioid compounds, by any route of administration, with known constipating activity on the gastrointestinal tract.

As used herein, “dosage unit” refers to physically discrete units suited as unitary dosages for the particular individual to be treated. Each unit may contain a predetermined quantity of active compound(s) calculated to produce the desired therapeutic effect(s) in association with the required pharmaceutical carrier. The specification for the dosage unit forms of the invention may be dictated by (a) the unique characteristics of the active compound(s) and the particular therapeutic effect(s) to be achieved, and (b) the limitations inherent in the art of compounding such active compound(s).

As used herein, “pharmaceutically acceptable” refers to those compounds, materials, compositions, and/or dosage forms that are, within the scope of sound medical judgment, suitable for contact with the tissues of human beings and animals without excessive toxicity, irritation, allergic response, or other problem complications commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. These physiologically acceptable salts are prepared by methods known in the art, e.g., by dissolving the free amine bases with an excess of the acid in aqueous alcohol, or neutralizing a free carboxylic acid with an alkali metal base such as a hydroxide, or with an amine.

Compounds described herein throughout, can be used or prepared in alternate forms. For example, many amino-containing compounds can be used or prepared as an acid addition salt. Often such salts improve isolation and handling properties of the compound. For example, depending on the reagents, reaction conditions and the like, compounds as described herein can be used or prepared, for example, as their hydrochloride or tosylate salts. Isomorphic crystalline forms, all chiral and racemic forms, N-oxide, hydrates, solvates, and acid salt hydrates, are also contemplated to be within the scope of the present invention.

Certain acidic or basic compounds of the present invention may exist as zwitterions. All forms of the compounds, including free acid, free base and zwitterions, are contemplated to be within the scope of the present invention. It is well known in the art that compounds containing both amino and carboxyl groups often exist in equilibrium with their zwitterionic forms. Thus, any of the compounds described herein throughout that contain, for example, both amino and carboxyl groups, also include reference to their corresponding zwitterions.

As used herein, “patient” refers to animals, including mammals, preferably humans.

As used herein, “prodrug” refers to compounds specifically designed to maximize the amount of active species that reaches the desired site of reaction that are of themselves typically inactive or minimally active for the activity desired, but through biotransformation are converted into biologically active metabolites.

As used herein, “stereoisomers” refers to compounds that have identical chemical constitution, but differ as regards the arrangement of the atoms or groups in space.

As used herein, “N-oxide” refers to compounds wherein the basic nitrogen atom of either a heteroaromatic ring or tertiary amine is oxidized to give a quaternary nitrogen bearing a positive formal charge and an attached oxygen atom bearing a negative formal charge.

As used herein, “hydrate” refers to a compound of the present invention which is associated with water in the molecular form, i.e., in which the H—OH bond is not split, and may be represented, for example, by the formula R.H₂O, where R is a compound of the invention. A given compound may form more than one hydrate including, for example, monohydrates (R.H₂O) or polyhydrates (R.nH₂O wherein n is an integer >1) including, for example, dihydrates (R.2H₂O), trihydrates (R.3H₂O), and the like, or hemihydrates, such as, for example, R.n/2H₂O, R.n/3H₂O, R.n/4H₂O and the like wherein n is an integer.

As used herein, “solvate” refers to a compound of the present invention which is associated with solvent in the molecular form, i.e., in which the solvent is coordinatively bound, and may be represented, for example, by the formula R.(solvent), where R is a compound of the invention. A given compound may form more than one solvate including, for example, monosolvates (R.(solvent)) or polysolvates (R.n(solvent)) wherein n is an integer>1) including, for example, disolvates (R.2(solvent)), trisolvates (R.3(solvent)), and the like, or hemisolvates, such as, for example, R.n/2(solvent), R.n/3(solvent), R.n/4(solvent) and the like wherein n is an integer. Solvents herein include mixed solvents, for example, methanol/water, and as such, the solvates may incorporate one or more solvents within the solvate.

As used herein, “acid salt hydrate” refers to a complex that may be formed through association of a compound having one or more base moieties with at least one compound having one or more acid moieties or through association of a compound having one or more acid moieties with at least one compound having one or more base moieties, said complex being further associated with water molecules so as to form a hydrate, wherein said hydrate is as previously defined and R represents the complex herein described above.

When any variable occurs more than one time in any constituent or in any formula, its definition in each occurrence is independent of its definition at every other occurrence. Combinations of substituents and/or variables are permissible only if such combinations result in stable compounds.

The piperidines derivatives useful in the methods of the invention as illustrated in formula (IA) can occur as the trans and cis stereochemical isomers at the 3- and 4-positions of the piperidine ring. The term “trans” as used herein refers, for example, in formula (IA) to the R substituent being on the opposite side of the R⁴ substituent, whereas in the “cis” isomer, the R² substituent and the R⁴ substituent are on the same side of the ring. The present invention contemplates the individual stereoisomers, as well as racemic mixtures. In the most preferred compounds of formula (IA), the R² substituent and the R⁴ substituent are in the “trans” orientation on the piperidine.

In addition to the “cis” and “trans” orientation of the R² substituent and the R⁴ substituent of formula (IA), the absolute stereochemistry of the carbon atoms bearing R² substituent and the R⁴ substituent of formula (IA) is also defined as using the commonly employed “R” and “S” definitions (Orchin et al., The Vocabulary of Organic Chemistry, John Wiley and Sons, Inc., 1981, page 126, which is incorporated herein by reference). The preferred compounds of the present invention are in which the configuration of both the R² substituent and the R⁴ substituents of formula (IA) on the piperidine ring are “R.”

Furthermore, asymmetric carbon atoms may be introduced into the molecule depending on the structure of R⁴. As such, these classes of compounds can exist as the individual “R” or “S” stereoisomers at these chiral centers, or the racemic mixture of the isomers, and all are contemplated as within the scope of the present invention. Preferably, a substantially pure stereoisomer of the compounds of this invention is used, i.e., an isomer in which the configuration at the chiral center is “R” or “S”, i.e., those compounds in which the configuration at the three chiral centers are preferably 3R, 4R, S or 3R, 4R, R.

As used herein, “peripheral” or “peripherally-acting” refers to an agent that acts outside of the central nervous system.

As used herein, “centrally-acting” refers to an agent that acts within the central nervous system (CNS).

In certain preferred embodiments, the methods may involve a peripheral opioid antagonist compound. The term “peripheral” designates that the compound acts primarily on physiological systems and components external to the central nervous system. In preferred form, the peripheral opioid antagonist compounds employed in the methods of the present invention exhibit high levels of activity with respect to peripheral tissue, such as, gastrointestinal tissue, while exhibiting reduced, and preferably substantially no, CNS activity. The phrase “substantially no CNS activity,” as used herein, means that less than about 20% of the pharmacological activity of the compounds employed in the present methods is exhibited in the CNS, preferably less than about 15%, more preferably less than about 10%, even more preferably less than about 5% and most preferably 0% of the pharmacological activity of the compounds employed in the present methods is exhibited in the CNS.

Furthermore, it is preferred in certain embodiments of the invention that the compound of formula (IA) does not substantially cross the blood-brain barrier and thereby interfere with the receptors in the CNS. The phrase “does not substantially cross,” as used herein, means that less than about 20% by weight of the compound employed in the present methods crosses the blood-brain barrier, preferably less than about 15% by weight, more preferably less than about 10% by weight, even more preferably less than about 5% by weight and most preferably 0% by weight of the compound crosses the blood-brain barrier.

The methods of the present invention are directed to treating and preventing non-opioid induced gastrointestinal dysfunction, including chronic constipation, slow colonic transit, low stool frequency, and poor stool consistency, with reduced undesirable side effects. Included among the conditions that may be treated in accordance with the methods of the invention is constipation associated with irritable bowel syndrome. In certain preferred embodiments, the methods are useful for the treatment and prevention of slow bowel transit but do not substantially affect the oral-cecal transit time.

The methods of the present invention may further employ one or more other active ingredients that may be conventionally employed in preventing or treating gastrointestinal dysfunction. Such conventional ingredients include, for example, laxatives, fiber, stool softeners, or bowel stimulants. Typical or conventional ingredients that may be included in the opioid component are described, for example, in the Physicians' Desk Reference, 2003, the disclosure of which is hereby incorporated herein by reference, in its entirety. Other optional components that may be employed in the methods and compositions of the present invention, in addition to those exemplified above, would be readily apparent to one of ordinary skill in the art, once armed with the teachings of the present disclosure.

Suitable 4-aryl-piperidine derivatives and a stereoisomer, a prodrug, a pharmaceutically acceptable salt, a hydrate, a solvate, an acid salt hydrate, an N-oxide and an isomorphic crystalline form thereof. Preferred 4-aryl-piperidine derivatives include, for example, the compounds disclosed in U.S. Pat. No. 5,250,542; U.S. Pat. No. 5,159,081; U.S. Pat. No. 5,270,328; and U.S. Pat. No. 5,434,171, U.S. Pat. No. 6,451,806 and U.S. Pat. No. 6,469,030, the disclosures of which are hereby incorporated herein by reference, in their entireties.

Accordingly, the present invention is directed, in part, to methods of treating or preventing non-opioid induced gastrointestinal dysfunction, comprising the step of:
administering to a patient in need thereof about 0.5 mg/day to about 18 mg/day of at least one 4-aryl-piperidine derivative or a stereoisomer, a prodrug, a pharmaceutically acceptable salt, a hydrate, a solvate, an acid salt hydrate, an N-oxide or an isomorphic crystalline form thereof;
wherein said patient is not receiving chronic or periodic exogenous opioids;
wherein said gastrointestinal dysfunction is chronic constipation, slow colonic transit, low stool frequency, poor stool consistency, or combinations thereof; and
wherein said 4-aryl-piperidine derivative is a compound of formula (IA): 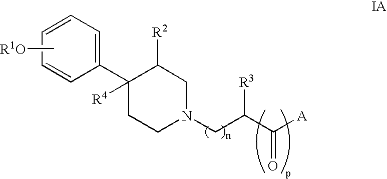
   wherein:
R¹ is hydrogen or alkyl;
R²is hydrogen, alkyl or alkenyl;
R³ is hydrogen, alkyl, alkenyl, aryl, cycloalkyl, cycloalkenyl, cycloalkyl-substituted alkyl, cycloalkenyl-substituted alkyl or aralkyl;
R⁴ is hydrogen, alkyl or alkenyl;
A is OR⁵ or NR⁶R⁷;
R⁵ is hydrogen, alkyl, alkenyl, cycloalkyl, cycloalkenyl, cycloalkyl-substituted alkyl, cycloalkenyl-substituted alkyl, or aralkyl;
R⁶ is hydrogen or alkyl;
R⁷ is hydrogen, alkyl, alkenyl, cycloalkyl, aryl, cycloalkyl-substituted alkyl, cycloalkenyl, cycloalkenyl-substituted alkyl, aralkyl, B, or alkylene substituted B or, together with the nitrogen atom to which they are attached, R⁶ and R⁷ form a heterocyclic ring;
B is 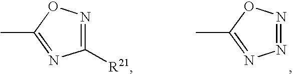
C(═O)W or NR⁸R⁹;
R⁸ is hydrogen or alkyl;
R⁹ is hydrogen, alkyl, alkenyl, cycloalkyl-substituted alkyl, cycloalkyl, cycloalkenyl, cycloalkenyl-substituted alkyl, aryl or aralkyl or, together with the nitrogen atom to which they are attached, R⁸ and R⁹ form a heterocyclic ring;
W is OR¹⁰, NR¹¹R¹², or OE;
R¹⁰ is hydrogen, alkyl, alkenyl, cycloalkyl, cycloalkenyl, cycloalkyl-substituted alkyl, cycloalkenyl-substituted alkyl, or aralkyl;
R¹¹ is hydrogen or alkyl;
R¹² is hydrogen, alkyl, alkenyl, aryl, cycloalkyl, cycloalkenyl, cycloalkyl-substituted alkyl, cycloalkenyl-substituted alkyl, aralkyl or alkylene substituted C(═O)Y or, together with the nitrogen atom to which they are attached, R¹¹ and R¹² form a heterocyclic ring;
E is 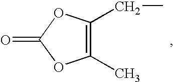
alkylene substituted (C═O)D, or —R¹³OC(═O)R¹⁴;
R¹³ is alkyl substituted alkylene;
R¹⁴ is alkyl;
D is OR¹⁵ or NR¹⁶R¹⁷;
R¹⁵ is hydrogen, alkyl, alkenyl, cycloalkyl, cycloalkenyl, cycloalkyl-substituted alkyl, cycloalkenyl-substituted alkyl, or aralkyl;
R¹⁶ is hydrogen, alkyl, alkenyl, aryl, aralkyl, cycloalkyl, cycloalkenyl, cycloalkyl-substituted alkyl or cycloalkenyl-substituted alkyl;
R¹⁷ is hydrogen or alkyl or, together with the nitrogen atom to which they are attached, R¹⁶ and R¹⁷ form a heterocyclic ring;
Y is OR¹⁸ or NR¹⁹R²⁰;
R¹⁸ is hydrogen, alkyl, alkenyl, cycloalkyl, cycloalkenyl, cycloalkyl-substituted alkyl, cycloalkenyl-substituted alkyl, or aralkyl;
R¹⁹ is hydrogen or alkyl;
R²⁰ is hydrogen, alkyl, alkenyl, aryl, cycloalkyl, cycloalkenyl, cycloalkyl-substituted alkyl, cycloalkenyl-substituted alkyl, or aralkyl or, together with the nitrogen atom to which they are attached, R¹⁹ and R²⁰ form a heterocyclic ring;
R²¹ is hydrogen or alkyl;
n is 0 to 4;
p is 0 or 1; and
provided that R¹⁰ is not hydrogen, when R¹ is hydrogen, R² is methyl, R³ is cycloalkyl-substituted alkyl, and R⁴ is methyl; and
provided that R¹⁰ is not alkyl, when R¹ is hydrogen, R² is methyl, R³ is aralkyl, and R⁴ is methyl.

In preferred embodiments, the compound of formula (IA) is a trans 3,4-isomer.

In certain embodiments employing compounds of formula (IA), it is preferred that
R¹ is hydrogen;
R²is alkyl;
n is 1 or 2;
R³ is benzyl, phenyl, cyclohexyl, or cyclohexylmethyl; and
R⁴ is alkyl.

In certain embodiments employing compounds of formula (IA), it is preferred that
A is OR⁵; and
R⁵ is hydrogen or alkyl.

In certain embodiments employing compounds of formula (IA), it is preferred that
A is NR⁶R⁷;
R⁶ is hydrogen;
R⁷ is alkylene substituted B; and
B is C(O)W.

In certain embodiments employing compounds of formula (IA), it is preferred that
R⁷ is (CH₂)_{q}—B;
q is about 1 to about 3;
W is OR¹⁰; and
R¹⁰ is hydrogen, alkyl, phenyl-substituted alkyl, cycloalkyl or cycloalkyl-substituted alkyl.

In certain embodiments including compounds of formula (IA), it is preferred that
W is NR¹¹R¹²
R¹¹ is hydrogen or alkyl; and
R¹² is hydrogen, alkyl or alkylene substituted C(═O)Y.

In certain embodiments employing compounds of formula (IA), it is preferred that
R¹² is (CH₂)ₘC(O)Y;
m is 1 to 3;
Y is OR¹⁸ or NR¹⁹R²⁰; and
R¹⁸, R¹⁹ and R²⁰ are independently hydrogen or alkyl.

In certain embodiments employing compounds of formula (IA), it is preferred that
W is OE;
E is CH₂C(═O)D;
D is OR¹⁵ or NR¹⁶R¹⁷;
R¹⁵ is hydrogen or alkyl;
R¹⁶ is methyl or benzyl; and
R¹⁷ is hydrogen.

In certain embodiments employing compounds of formula (IA), it is preferred that
W is OE;
E is R¹³C(═O)R¹⁴;
R¹³ is —CH(CH₃)— or —CH(CH₂CH₃)—; and
R¹⁴ is alkyl.

In certain embodiments including compounds of formula (IA), it is preferred that p is 1.

In certain embodiments employing compounds of formula (IA), it is preferred that the configuration at positions 3 and 4 of the piperidine ring is each R.

Preferred compounds of formula (IA) include:
Q-CH₂CH(CH₂(C₆H₅))C(O)OH,
Q-CH₂CH₂CH(C₆H₅)C(O)NHCH₂C(O)OCH₂CH₃,
Q-CH₂CH₂CH(C₆H₅)C(O)NHCH₂C(O)OH,
Q-CH₂CH₂CH(C₆H₅)C(O)NHCH₂C(O)NHCH₃,
Q-CH₂CH₂CH(C₆H₅)C(O)NHCH₂C(O)NHCH₂CH₃,
G-NH(CH₂)₂C(O)NH₂,
G-NH(CH₂)₂C(O)NHCH₃,
G-NHCH₂C(O)NH₂,
G-NHCH₂C(O)NHCH₃,
G-NHCH₂C(O)NHCH₂CH₃,
G-NH(CH₂)₃C(O)OCH₂CH₃,
G-NH(CH₂)₃C(O)NHCH₃,
G-NH(CH₂)₂C(O)OH,
G-NH(CH₂)₃C(O)OH,
Q-CH₂CH(CH₂(C₆H₁₁))C(O)NH(CH₂)₂C(O)OH,
Q-CH₂CH(CH₂(C₆H₁₁))C(O)NH(CH₂)₂C(O)NH₂,
Z-NHCH₂C(O)OH,
Z-NHCH₂C(O)NH₂,
Z-NHCH₂C(O)N(CH₃)₂,
Z-NHCH₂C(O)NHCH(CH₃)₂,
Z-NH(CH₂)₂C(O)OCH₂(C₆H₅),
Z-NH(CH₂)₂C(O)NHCH₂CH₃,
Z-NH(CH₂)₃C(O)NHCH₃,
Z-NHCH₂C(O)NHCH₂C(O)OH,
Z-NHCH₂C(O)OCH₂C(O)OCH₃,
Z-NHCH₂C(O)OCH₂C(O)NHCH₃,
Z-NHCH₂C(O)O-(4-methoxycyclohexyl),
Z-NHCH₂C(O)OCH₂C(O)NHCH₂(C₆H₅) and
Z-NHCH₂C(O)OCH(CH₃)OC(O)CH₃;
   wherein:

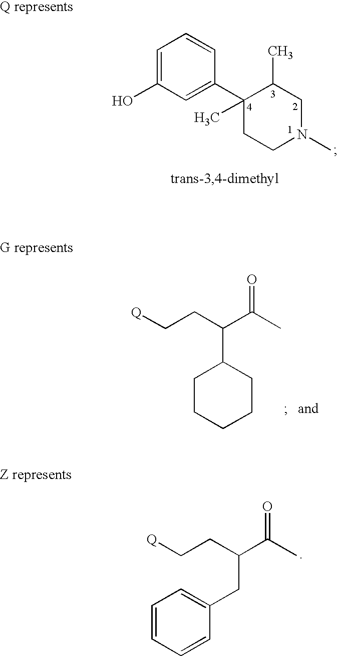

More preferred compounds of formula (IA) include:
(+)-Z-NHCH₂C(O)OH,
(−)-Z-NHCH₂C(O)OH,
(3R,4R)-Z-NHCH₂C(O)NHCH₂(C₆H₅) and
(3R,4R)-G-NH(CH₂)₃C(O)OH,
   wherein Q, Z and G are as defined above. Even more preferred compounds of formula (IA) include (+)-Z-NHCH₂C(O)OH and (−)-Z-NHCH₂C(O)OH, most especially (+)-Z-NHCH₂C(O)OH, where Z is as defined above.

Even more preferred compounds of formula (IA) include Q-CH₂CH(CH₂(C₆H₅))C(O)OH, wherein Q is as defined above. It is especially preferred when said compound is (3R, 4R, S)-Q-CH₂CH(CH₂(C₆H₅))C(O)OH.

A particularly preferred embodiment of the present invention is the compound (+)-Z-NHCH₂C(O)OH, i.e., the compound of the following formula (II):

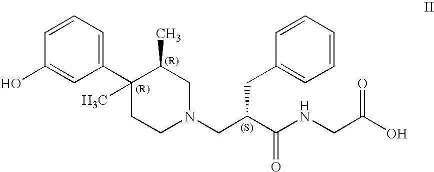

The compound of formula (II) has low solubility in water except at low or high pH conditions. Zwitterionic character may be inherent to the compound, and may impart desirable properties such as poor systemic absorption and sustained local affect on the gut following oral administration.

In especially preferred embodiments, the compound of a formula (IA) is a substantially pure stereoisomer.

In addition, the present invention is directed, in part, to methods of treating or preventing non-opioid induced gastrointestinal dysfunction, comprising the step of:
administering to a patient in need thereof about 0.5 mg/day to about 18 mg/day of at least one 4-aryl-piperidine derivative or a stereoisomer, a prodrug, a pharmaceutically acceptable salt, a hydrate, a solvate, an acid salt hydrate, an N-oxide or an isomorphic crystalline form thereof;
wherein said patient is not receiving chronic or periodic exogenous opioids;
wherein said gastrointestinal dysfunction is chronic constipation, slow colonic transit, low stool frequency, poor stool consistency, or combinations thereof; and
wherein said 4-aryl-piperidine derivative is [[2-[[-4-(3-hydroxyphenyl)-3,4-dimethylpiperidin-1-yl]methyl]-3-phenylpropanoyl]amino]acetic acid. Preferably, the [[2-[[-4-(3-hydroxyphenyl)-3,4-dimethylpiperidin-1-yl]methyl]-3-phenylpropanoyl]amino]acetic acid is in hydrate form, more preferably, [[2-[[-4-(3-hydroxyphenyl)-3,4-dimethylpiperidin-1-yl]methyl]-3-phenylpropanoyl]amino]acetic acid dihydrate, even more preferably in substantially pure isomeric form, most especially [[(2S)-2-[[(3R,4R)-4-(3-hydroxyphenyl)-3,4-dimethylpiperidin-1-yl]methyl]-3-phenylpropanoyl]amino]acetic acid dihydrate (alvimopan).

In accordance with the methods of the present invention, the 4-aryl piperidine compounds may be administered to a patient in a dosage range of from about 0.5 mg/day to about 18 mg/day (and all combinations and subcombinations of dosage ranges and specific dosages therein).

In certain preferred embodiments of the invention, said 4-aryl-piperidine derivative or stereoisomer, prodrug, pharmaceutically acceptable salt, hydrate, solvate, acid salt hydrate, N-oxide or isomorphic crystalline form thereof is administered at a level of at least about 0.75 mg/day, more preferably at a level of at least about 1 mg/day, even more preferably at a level of at least about 2 mg/day, and yet even more preferably at a level of at least about 3 mg/day.

In certain preferred embodiments of the invention, said 4-aryl-piperidine derivative or stereoisomer, prodrug, pharmaceutically acceptable salt, hydrate, solvate, acid salt hydrate, N-oxide or isomorphic crystalline form thereof is administered at a level of less than about 15 mg/day, more preferably at a level of less than about 12 mg/day, even more preferably at a level of less than about 9 mg/day, and yet even more preferably at a level of less than about 6 mg/day.

In certain particularly preferred embodiments where gastrointestinal dysfunction is chronic constipation, including chronic constipation is associated with irritable bowel syndrome, the 4-aryl-piperidine derivative, preferably alvimopan, or stereoisomer, prodrug, pharmaceutically acceptable salt, hydrate, solvate, acid salt hydrate, N-oxide or isomorphic crystalline form thereof is administered at a level of about 0.5 mg/day to about 10 mg/day.

In certain preferred embodiments of the invention, said 4-aryl-piperidine derivative or stereoisomer, prodrug, pharmaceutically acceptable salt, hydrate, solvate, acid salt hydrate, N-oxide or isomorphic crystalline form thereof is administered for at least about 1 day, more preferably for at least about 2 days, even more preferably for at least about 3 days, yet even more preferably, for at least about 5 days, and further more preferably, for at least about 7 days.

The compounds employed in the methods of the present invention may exist in prodrug form. As used herein, “prodrug” is intended to include any covalently bonded carriers that release the active parent drug, for example, as according to formulas (IA) or other formulas or compounds employed in the methods of the present invention in vivo when such prodrug is administered to a mammalian subject. Since prodrugs are known to enhance numerous desirable qualities of pharmaceuticals (e.g., solubility, bioavailability, manufacturing, etc.) the compounds employed in the present methods may, if desired, be delivered in prodrug form. Thus, the present invention contemplates methods of delivering prodrugs. Prodrugs of the compounds employed in the present invention, for example formula (IA), may be prepared by modifying functional groups present in the compound in such a way that the modifications are cleaved, either in routine manipulation or in vivo, to the parent compound.

Accordingly, prodrugs include, for example, compounds described herein in which a hydroxy, amino, or carboxy group is bonded to any group that, when the prodrug is administered to a mammalian subject, cleaves to form a free hydroxyl, free amino, or carboxylic acid, respectively. Examples include, but are not limited to, acetate, formate and benzoate derivatives of alcohol and amine functional groups; and alkyl, carbocyclic, aryl, and alkylaryl esters such as methyl, ethyl, propyl, iso-propyl, butyl, isobutyl, sec-butyl, tert-butyl, cyclopropyl, phenyl, benzyl, and phenethyl esters, and the like.

The compounds employed in the methods of the present invention may be prepared in a number of ways well known to those skilled in the art. The compounds can be synthesized, for example, using the methods described in U.S. Pat. No. 5,250,542, U.S. Pat. No. 6,469,030, and U.S. Pat. No. 6,451,806, the disclosures of which are hereby incorporated by reference, in their entireties. All processes disclosed in association with the present invention are contemplated to be practiced on any scale, including milligram, gram, multigram, kilogram, multikilogram or commercial industrial scale.

As discussed in detail above, compounds employed in the present methods may contain one or more asymmetrically substituted carbon atoms, and may be isolated in optically active or racemic forms. Thus, all chiral, diastereomeric, racemic forms and all geometric isomeric forms of a structure are intended, unless the specific stereochemistry or isomeric form is specifically indicated. It is well known in the art how to prepare and isolate such optically active forms. For example, mixtures of stereoisomers may be separated by standard techniques including, but not limited to, resolution of racemic forms, normal, reverse-phase, and chiral chromatography, preferential salt formation, recrystallization, and the like, or by chiral synthesis either from chiral starting materials or by deliberate synthesis of target chiral centers.

As will be readily understood, functional groups present may contain protecting groups during the course of synthesis. Protecting groups are known per se as chemical functional groups that can be selectively appended to and removed from functionalities, such as hydroxyl groups and carboxyl groups. These groups are present in a chemical compound to render such functionality inert to chemical reaction conditions to which the compound is exposed. Any of a variety of protecting groups may be employed with the present invention. Preferred protecting groups include the benzyloxycarbonyl group and the tert-butyloxycarbonyl group. Other preferred protecting groups that may be employed in accordance with the present invention may be described in Greene, T. W. and Wuts, P. G. M., Protective Groups in Organic Synthesis 2d. Ed., Wiley & Sons, 1991.

As noted above, the compounds of the present invention can exist as the individual stereoisomers. Preferably, reaction conditions are adjusted as disclosed in U.S. Pat. No. 4,581,456 or as set forth in Example 1 of U.S. Pat. No. 5,250,542 to be substantially stereoselective and provide a racemic mixture of essentially two enantiomers. These enantiomers may then be resolved. A procedure which may be employed to prepare the resolved starting materials used in the synthesis of these compounds includes treating a racemic mixture of alkyl-3,4-dimethyl-4-(3-alkoxyphenyl)piperidine with either (+)- or (−)-ditoluoyl tartaric acid to provide the resolved intermediate. This compound may then be dealkylated at the 1-position with vinyl chloroformate and finally converted to the desired 4-(3-hydroxyphenyl)piperidine isomer.

As will be understood by those skilled in the art, the individual enantiomers of the invention can also be isolated with either (+) or (−) dibenzoyl tartaric acid, as desired, from the corresponding racemic mixture of the compounds of the invention. Preferably, the (+)-trans enantiomer is obtained.

Although the (+)trans-3,4 stereoisomer is preferred, all of the possible stereoisomers of the compounds described herein are within the contemplated scope of the present invention. Racemic mixtures of the stereoisomers as well as the substantially pure stereoisomers are within the scope of the invention. The term “substantially pure,” as used herein, refers to at least about 90 mole percent, more preferably at least about 95 mole percent and most preferably at least about 98 mole percent of the desired stereoisomer is present relative to other possible stereoisomers.

Another synthetic route can involve the reaction of a substituted piperidine with a haloalkylnitrile. The nitrile group of the resulting piperidine alkylnitrile can be hydrolyzed to the corresponding carboxylic acid.

The compounds employed in the methods of the present invention may be administered by any means that results in the contact of the active agents with the agents' site or site(s) of action in the body of a patient. The compounds may be administered by any conventional means available for use in conjunction with pharmaceuticals, either as individual therapeutic agents or in a combination of therapeutic agents. For example, they may be administered as the sole active agents in a pharmaceutical composition, or they can be used in combination with other therapeutically active ingredients.

The compounds are preferably combined with a pharmaceutical carrier selected on the basis of the chosen route of administration and standard pharmaceutical practice as described, for example, in Remington's Pharmaceutical Sciences (Mack Publishing Co., Easton, Pa., 1980), the disclosures of which is hereby incorporated herein by reference, in its entirety.

Compounds of the present invention can be administered to a mammalian host in a variety of forms adapted to the chosen route of administration, e.g., orally. Other acceptable routes of administration are parenteral including intravenous; transepithelial including transdermal, transnasal, ophthalmic, sublingual and buccal; topical including ophthalmic, dermal, ocular, and rectal; nasal or pulmonary inhalation via insufflation or aerosol; and rectal systemic.

The active compound may be orally administered, for example, with an inert diluent or with an assimilable edible carrier, it may be enclosed in hard or soft shell gelatin capsules, it may be compressed into tablets, or it may be incorporated directly with the food of the diet. For oral therapeutic administration, the active compound may be incorporated with excipient and used in the form of ingestible tablets, buccal tablets, troches, capsules, elixirs, suspensions, syrups, wafers, and the like. The amount of active compound(s) in such therapeutically useful compositions is from about 0.5 mg/day to about 18 mg/day of active compound.

In certain preferred embodiments of the invention, said 4-aryl-piperidine derivative or stereoisomer, prodrug, pharmaceutically acceptable salt, hydrate, solvate, acid salt hydrate, N-oxide or isomorphic crystalline form thereof is administered at a level of at least about 0.75 mg/day, more preferably at a level of at least about 1 mg/day, even more preferably at a level of at least about 2 mg/day, and yet even more preferably at a level of at least about 3 mg/day.

In certain preferred embodiments of the invention, said 4-aryl-piperidine derivative or stereoisomer, prodrug, pharmaceutically acceptable salt, hydrate, solvate, acid salt hydrate, N-oxide or isomorphic crystalline form thereof is administered at a level of less than about 15 mg/day, more preferably at a level of less than about 12 mg/day, even more preferably at a level of less than about 9 mg/day, and yet even more preferably at a level of less than about 6 mg/day.

In certain preferred embodiments of the invention, said 4-aryl-piperidine derivative or stereoisomer, prodrug, pharmaceutically acceptable salt, hydrate, solvate, acid salt hydrate, N-oxide or isomorphic crystalline form thereof is administered for at least about 1 day, more preferably for at least about 2 days, even more preferably for at least about 3 days, yet even more preferably, for at least about 5 days, and further more preferably, for at least about 7 days.

The tablets, troches, pills, capsules and the like may also contain one or more of the following: a binder, such as gum tragacanth, acacia, corn starch or gelatin; an excipient, such as dicalcium phosphate; a disintegrating agent, such as corn starch, potato starch, alginic acid and the like; a lubricant, such as magnesium stearate; a sweetening agent such as sucrose, lactose or saccharin; or a flavoring agent, such as peppermint, oil of wintergreen or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavoring, such as cherry or orange flavor. Of course, any material used in preparing any dosage unit form is preferably pharmaceutically pure and substantially non-toxic in the amounts employed. In addition, the active compound may be incorporated into sustained-release preparations and formulations.

The active compound may also be administered parenterally. Solutions of the active compounds as free bases or pharmacologically acceptable salts can be prepared in water suitably mixed with a surfactant, such as hydroxypropylcellulose. A dispersion can also be prepared in glycerol, liquid polyethylene glycols and mixtures thereof and in oils. Under ordinary conditions of storage and use, these preparations may contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. In all cases, the form is preferably sterile and fluid to provide easy syringability. It is preferably stable under the conditions of manufacture and storage and is preferably preserved against the contaminating action of microorganisms such as bacteria and fungi. The carrier may be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating, such as lecithin, by the maintenance of the required particle size in the case of a dispersion, and by the use of surfactants. The prevention of the action of microorganisms may be achieved by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions may be achieved by the use of agents delaying absorption, for example, aluminum monostearate and gelatin.

Sterile injectable solutions may be prepared by incorporating the active compounds in the required amounts, in the appropriate solvent, with various of the other ingredients enumerated above, as required, followed by filtered sterilization. Generally, dispersions may be prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation may include vacuum drying and the freeze-drying technique that yield a powder of the active ingredient, plus any additional desired ingredient from the previously sterile-filtered solution thereof.

The therapeutic compounds of this invention may be administered to a patient alone or in combination with a pharmaceutically acceptable carrier. As noted above, the relative proportions of active ingredient and carrier may be determined, for example, by the solubility and chemical nature of the compounds, chosen route of administration and standard pharmaceutical practice.

The dosage of the compounds of the present invention that will be most suitable for prophylaxis or treatment will vary with the form of administration, the particular compound chosen and the physiological characteristics of the particular patient under treatment. Generally, small dosages may be used initially and, if necessary, increased by small increments until the desired effect under the circumstances is reached. Generally speaking, oral administration may require higher dosages.

The combination products useful in the methods of this invention, such as pharmaceutical compositions comprising 4-aryl-piperidine derivatives with additional active ingredients, may be in any dosage form, such as those described herein, and can also be administered in various ways, as described herein. In a preferred embodiment, the combination products of the invention are formulated together, in a single dosage form (that is, combined together in one capsule, tablet, powder, or liquid, etc.). When the combination products are not formulated together in a single dosage form, the 4-aryl-piperidine derivative and additional active ingredient may be administered at the same time or simultaneously (that is, together), or in any order. When not administered at the same time or simultaneously, that is, when administered sequentially, preferably the administration of a 4-aryl-piperidine derivative and additional active ingredient occurs less than about one hour apart, more preferably less than about 30 minutes apart, even more preferably less than about 15 minutes apart, and still more preferably less than about 5 minutes apart.

Preferably, administration of the combination products of the invention is oral, although other routes of administration, as described above, are contemplated to be within the scope of the present invention. Although it is preferable that the 4-aryl-piperidine derivative and the additional active ingredients are all administered in the same fashion (that is, for example, both orally), if desired, they may each be administered in different fashions (that is, for example, one component of the combination product may be administered orally, and another component may be administered intravenously). The dosage of the combination products of the invention may vary depending upon various factors such as the pharmacodynamic characteristics of the particular agent and its mode and route of administration, the age, health and weight of the recipient, the nature and extent of the symptoms, the kind of concurrent treatment, the frequency of treatment, and the effect desired.

Particularly when provided as a single dosage form, the potential exists for a chemical interaction between the combined active ingredients. For this reason, the preferred dosage forms of the combination products of this invention are formulated such that although the active ingredients are combined in a single dosage form, the physical contact between the active ingredients is minimized (that is, reduced).

In order to minimize contact, one embodiment of this invention where the product is orally administered provides for a combination product wherein one active ingredient is enteric coated. By enteric coating one or more of the active ingredients, it is possible not only to minimize the contact between the combined active ingredients, but also, it is possible to control the release of one of these components in the gastrointestinal tract such that one of these components is not released in the stomach but rather is released in the intestines. Another embodiment of this invention where oral administration is desired provides for a combination product wherein one of the active ingredients is coated with a sustained-release material that effects a sustained-release throughout the gastrointestinal tract and also serves to minimize physical contact between the combined active ingredients. Furthermore, the sustained-released component can be additionally enteric coated such that the release of this component occurs only in the intestine. Still another approach would involve the formulation of a combination product in which the one component is coated with a sustained and/or enteric release polymer, and the other component is also coated with a polymer such as a low-viscosity grade of hydroxypropyl methylcellulose (HPMC) or other appropriate materials as known in the art, in order to further separate the active components. The polymer coating serves to form an additional barrier to interaction with the other component.

Dosage forms of the combination products of the present invention wherein one active ingredient is enteric coated can be in the form of tablets such that the enteric coated component and the other active ingredient are blended together and then compressed into a tablet or such that the enteric coated component is compressed into one tablet layer and the other active ingredient is compressed into an additional layer. Optionally, in order to further separate the two layers, one or more placebo layers may be present such that the placebo layer is between the layers of active ingredients. In addition, dosage forms of the present invention can be in the form of capsules wherein one active ingredient is compressed into a tablet or in the form of a plurality of microtablets, particles, granules or non-pareils, which are then enteric coated. These enteric coated microtablets, particles, granules or non-pareils are then placed into a capsule or compressed into a capsule along with a granulation of the other active ingredient.

These as well as other ways of minimizing contact between the components of combination products of the present invention, whether administered in a single dosage form or administered in separate forms but at the same time by the same manner, will be readily apparent to those skilled in the art, once armed with the present disclosure.

Pharmaceutical kits useful in the methods of the invention are also within the ambit of the present invention. Sterilization of the container may be carried out using conventional sterilization methodology well known to those skilled in the art. The sterile containers of materials may comprise separate containers, or one or more multi-part containers, as exemplified by the UNIVIAL™ two-part container (available from Abbott Labs, Chicago, Ill.), as desired. The 4-aryl-piperidine derivative and the optional additional active ingredient may be separate, or combined into a single dosage form as described above. Such kits may further include, if desired, one or more of various conventional pharmaceutical kit components, such as for example, one or more pharmaceutically acceptable carriers, additional vials for mixing the components, etc., as will be readily apparent to those skilled in the art. Instructions, either as inserts or as labels, indicating quantities of the components to be administered, guidelines for administration, and/or guidelines for mixing the components, may also be included in the kit.

### EXAMPLES

The present invention will now be illustrated by reference to the following specific, non-limiting examples. The examples are not intended to limit the scope of the present invention.

### Example 1

Methodology:

This was an ascending dose level safety study (Phase I) intended to determine the frequency and severity of adverse events (AEs) and to estimate the maximum tolerated dose of alvimopan. Subjects were screened to ensure their status as healthy volunteers. They were only enrolled if they had no clinically significant abnormalities on history, physical, or laboratory examinations. Post study physical examination and laboratory tests, including serum chemistries and complete blood count, were performed.

Duration of Treatment:

Alvimopan or placebo was administered TID for 4 days.

Reference Therapy, Dose, and Mode of Administration:

One subject in each dose group was randomly assigned to receive placebo that was identical in appearance to the active study medication.

Results:

Forty-four subjects were randomized for treatment. One subject at each dose level received placebo (N=5); the rest received alvimopan. Alvimopan was generally well tolerated in doses up to 54 mg daily (18 mg TID) for 4 days. The incidence of gastrointestinal AEs increased with increasing dose of alvimopan. One or more incidence of mild or moderate “abdominal pain” was reported by 83-100% of subjects receiving 18 or 54 mg daily doses (6 or 18 mg TID), but was infrequent at lower doses (range 11-18%). One or more incidence of diarrhea was reported by 83% of subjects receiving 54 mg daily doses (18 mg TID), but was infrequent at lower doses (range 9-22%). One or more incidence of flatulence was reported by 50-78% of subjects receiving 18 or 54 mg daily doses (6 or 18 mg TID), but was less frequent at lower doses (range 18-33%). One subject receiving the highest dose, 54 mg/day (18 mg TID), withdrew during the second day of dosing due to adverse events of abdominal pain, nausea, and diarrhea.

### Example 2

A single-center, placebo-controlled, randomized, double-blind, balanced two period cross-over study of the effect of alvimopan on GI transit in subjects with functional constipation was carried out.

Subjects (n=24) meeting the initial screening criteria for functional constipation by history, and who had 3 or fewer stools during a 7-day screening period, were randomly assigned to receive initially either orally-administered alvimopan, 3 mg twice daily for 7 days or matching placebo. Following a two- to four-week washout period, subjects received the opposite treatment for a 7-day period. Subjects did not enter second period unless their bowel function had returned to baseline levels as subjectively assessed by the subject. Subjects' diets were not to be changed significantly while participating in the study.

Subjects underwent measurement of their whole bowel transit (radio-opaque markers) and oral-cecal transit (hydrogen-breath test) prior to receiving the first treatment (baseline) and at the end of each treatment period. During the screening and treatment periods, subjects recorded the time of each bowel movement (defecatory frequency), an assessment of each stool's consistency (using the Bristol Stool Form Scale), and an overall rating of their satisfaction with their bowel movements over each period (1-5 scale: 1=much worse than usual, 2=worse than usual, 3=no change, 4=better than usual, 5=much better than usual). Stool output per 7-day period was calculated from the weight of all stools collected during each treatment period (stools collected over the 7-day assessment period). Some studies have found that GI transit in females of child bearing potential varies depending on the phase of their menstrual cycle (females in the luteal phase of the menstrual cycle have slower transit than women in the follicular phase or men (Miller, et al., Dig. Dis. Sci., 1997, 42: 10-18; Wald, et al., Gastroenterology, 1981, 80: 1497-1500). Therefore, all such female subjects were studied during the 10 days following menstruation.

Study Population

Twenty-three subjects with a history of functional constipation and who met the inclusion and exclusion criteria were enrolled into the study. Since the primary end-point for this study focuses on gut motility, the functional constipation population eligible for inclusion in the study was low frequency of defecation (≦3 stools per week).

Study Assessments and Procedures

Screening

Screening was performed within 4 weeks of the first dose and included demography, medical examination, height, body-weight, vital signs, 12-lead ECG, laboratory safety screens, drugs of abuse screen, a serum pregnancy test, and serologic tests for Hw, Hepatitis B & C. Subjects provided written informed consent prior to screening. Subjects were instructed to record the time and day of all defecations for the 7-day period starting at 8:00 AM the morning subsequent to the screening visit until 8:00 AM one week later.

Baseline Transit Studies

Baseline bowel transit studies were performed (as outlined below) after subjects were determined to meet all entry criteria (including ≦3 bowel movements during the 7-day screening period).

Treatment Period Study Days 1-7

Each treatment period comprised seven days of dosing (oral alvimopan 3 mg or matching placebo, given at 8:00 AM and 8:00 PM). For each defecation, subjects recorded the time, date, and stool form (Bristol Stool Form Criteria) (Heaton, et al., Gut, 1991, 73-79). Subjects rated their overall satisfaction with their bowel movements for the treatment period on Day 7.

For Day 1 of each treatment period, the following procedures were conducted in the clinic prior to the morning dosing: a urine drug screen, and a urine/serum pregnancy test (when applicable), semi-supine (i.e., 45-degree angle, pillow, etc.) blood pressure and pulse rate measurements and recording of baseline signs and symptoms. All subjects received their first dose of alvimopan 3 mg or placebo after a light breakfast in the clinic at approximately 8 AM in the morning and according to the randomization schedule. Each dose of alvimopan or placebo was taken with 240 ml of still room-temperature water at each dosing occasion. Subjects were then dispensed the study drug to be taken until the evening dose on Day 5, as well as the Stizmark capsules containing the radio-opaque markers and discharged. Subjects were admitted to the study unit on the evening of Day 5 until completion of the transit studies on Day 7. They were asked to complete a medication diary with the times of morning and evening dosing as an outpatient, as well as any symptoms they experienced at home. Subjects collected all stools from Days 1 to 7. Subjects recorded the time, date, and stool form (Bristol Stool Form Criteria) (Heaton, et al., Gut, 1991, 73-79) for each defecation. Subjects rated their overall satisfaction with their bowel movements for the treatment period on Day 7.

Treatment Days 3-7

Whole Bowel Transit Measurement (WBT):

Whole bowel transit was determined by using a modification of the radio-opaque marker method developed by Metcalf et al., Gastroenterology, 1987, 92: 40-47. This method is reproducible and correlates well with scintigraphic methods of measuring colonic transit (Degen, et al., Gut, 1996, 39: 299-305). Subjects ingested, at 8:00 AM on the mornings of Days 3, 4, 5, one capsule containing 24 radio-opaque markers (Sitzmark; Konsyl Pharmaceuticals, Fort Worth, Tex.). Stools from Days 3 to 7 were collected and examined by x-ray for the presence of the radio-opaque markers. Subjects were admitted to the study unit on evening of Day 5 for the duration of the study period. A plain abdominal radiograph was obtained on Day 7 at 8:00 AM to determine the number and location of the markers in each of six locations in the colon. Weights were assigned to the six locations in the colon and a weighted average of the number of radio-opaque markers in the colon was calculated. For purposes of comparison, the method being used was identical to the method described by Barr, et al., in the evaluation of alvimopan's effect on whole bowel transit in subjects receiving oral morphine (Barr, et al., Clin. Pharmacol. Ther., 2000, 67: 91).

Oral-Cecal Transit Time (OCTT):

The transit time for mouth to cecum, a reflection of small bowel transit, was measured utilizing the hydrogen breath test. This method is based on the measurement of hydrogen in exhaled air that is produced when an orally administered, non-absorbable, disaccharide (lactulose) is fermented by colonic bacteria. The time between ingestion of lactulose (10 g) and the sustained increase in hydrogen in the breath (hydrogen concentration in end-expiratory breath samples as measured by gas chromatography) represents the oral-cecal transit time (OCTT). This test is reproducible (coefficient of variation within individuals of 8%), and correlates well (r=0.95) with scintigraphic methods of measuring small bowel transit time (Miller, et al., Dig. Dis. Sci., 1997, 42: 10-18; Casellas, et al., Digestion, 1998, 59: 696-702; Jorge, et al., Eur. J. Surg., 1994, 160: 409-16). Subjects ingested 10 g of lactulose at 9:00 AM on Day 7 of each treatment period. Breath samples were measured every 15 minutes following ingestion of the lactulose. Transit time was determined by the first time of three consecutive samples in which there was at least a doubling of the baseline breath hydrogen concentration. The OCTT test was conducted after the completion of the assessment of WBT.

Post-Treatment Follow-Up

Subjects were given a physical examination, vital signs, and blood and urine samples obtained for safety laboratory studies at the conclusion of their last treatment period. Any abnormal findings were followed until resolution.

Investigational Product(s)

Alvimopan and matched placebo were supplied as capsules in 1.5 mg doses. All subjects received a twice-daily oral dose of alvimopan or placebo according to the randomization schedule for 7 days.

Results

This single-center, randomized, placebo-controlled, crossover trial was designed to determine the effect of alvimopan on bowel transit of patients with chronic constipation. Whole bowel transit (WBT), bowel movement frequency as total bowel movement (BM), and spontaneous complete BM (SCBM), BM symptoms of straining, discomfort, and satisfaction were compared between placebo and alvimopan (3 mg BID) for 7 days. Safety and tolerability were assessed.

Twenty-three adult males and females with at least 6-month history of chronic constipation, who did not meet the Rome II criteria for IBS, and had <3 BM during a 7 day baseline period participated in this study. The following were performed for each test arm: WBT estimated by a weighted sum of retained radio-opaque markers (based on number and location in the colon on single x-ray 48 hours post marker ingestion) and mean colonic transit time (MCTT) calculated by the method of Metcalf et al., Gastroenterology, 1987, 92: 40-47; and bowel movement frequency (BMF) and symptoms by patient self-report diary card.

Mean (±SEM) BMF for all subjects measured during baseline was 2.6±1.0 and mean SCBM was 0.8±1.0. Alvimopan significantly (p<0.049) increased WBT 32% (109±17 v. 160±17 WSM). A corresponding 19% reduction in MCTT was also seen (56±6 v. 69±5 hours). This alvimopan versus placebo response over 7 days was reflected by mean increases above baseline for BMF (1.0 v. 0.5) and SCBM (0.6 v. 0.3). Alvimopan improved BM consistency, straining, discomfort, and satisfaction compared to placebo. Adverse events were generally mild in both arms with slightly higher incidence of GI-related adverse effects in the alvimopan arm. No subjects prematurely discontinued either treatment arm due to an adverse effect.

OCTT was not changed by administration of alvimopan v. placebo as measured by the lactulose hydrogen breath test, indicating that alvimopan appeared to not influence small intestinal transit time in this study.

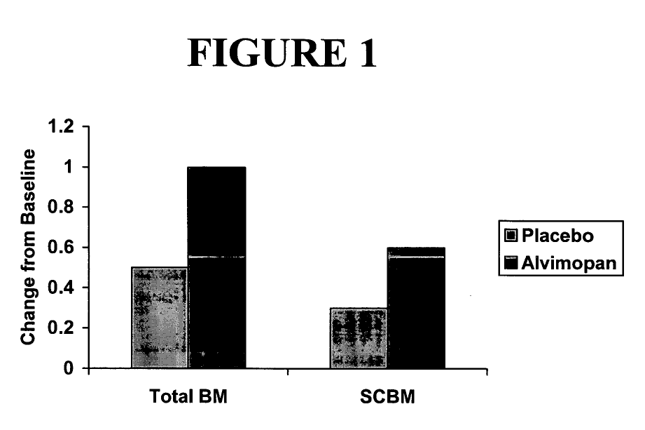

Alvimopan (3 mg BID) increased bowel transit as evidenced by increased WBT and decreased MCTT. Alvimopan improved BM frequency, stool hardness, straining, discomfort and satisfaction of bowel movements. The results are shown in FIGS. 1, 2, and 3.

FIG. 1 shows bowel movement frequency: bowel movement (BM) or spontaneous complete bowel movement (SCBM) per week (as a change from baseline). Alvimopan treatment increased both the mean total bowel movement increase from baseline and the mean SCBM change from baseline.

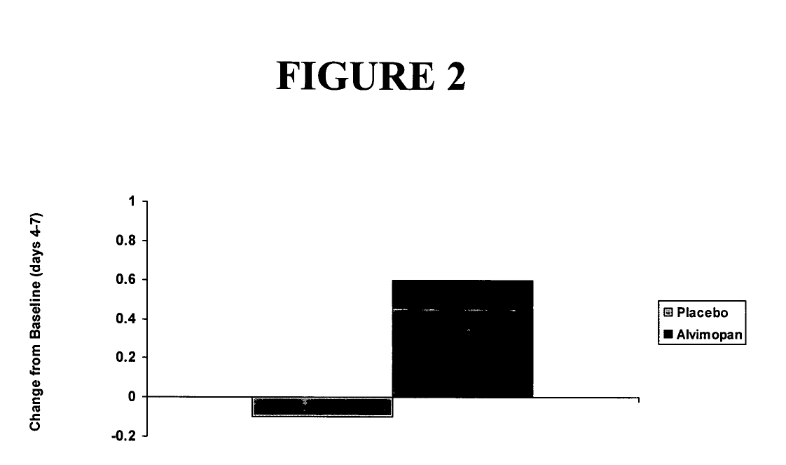

FIG. 2 shows stool consistency (with Bristol Stool Form Scale). Alvimopan treatment increased the mean bowel movement Bristol Stool Form Scale score indicating a change to a softer stool.

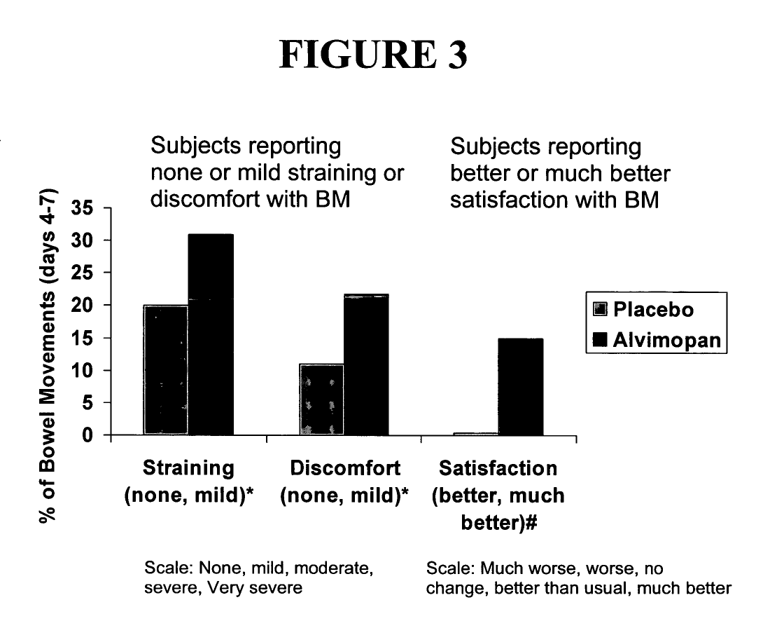

FIG. 3 shows bowel movement characteristics as percent of bowel movements, no/mild straining or discomfort and improved bowel movement satisfaction. Alvimopan treatment increased the % of bowel movements with no/mild straining or discomfort and improved satisfaction with bowel movements.

When ranges are used herein for physical properties, such as molecular weight, or chemical properties, such as chemical formulae, all combinations and subcombinations of ranges specific embodiments therein are intended to be included.

The disclosures of each patent, patent application, and publication cited or described in this document are hereby incorporated herein by reference, in their entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A method of treating or preventing non-opioid induced gastrointestinal dysfunction, comprising the step of:
administering to a patient in need thereof about 0.5 mg/day to about 18 mg/day of at least one 4-aryl-piperidine derivative or a stereoisomer, a prodrug, a pharmaceutically acceptable salt, a hydrate, a solvate, an acid salt hydrate, an N-oxide or an isomorphic crystalline form thereof;
wherein said patient is not receiving chronic or periodic exogenous opioids;
wherein said gastrointestinal dysfunction is chronic constipation, slow colonic transit, low stool frequency, poor stool consistency, or combinations thereof; and
wherein said 4-aryl-piperidine derivative is [[2-[[-4-(3-hydroxyphenyl)-3,4-dimethylpiperidin-1-yl]methyl]-3-phenylpropanoyl]amino]acetic acid.

2. A method according to claim 1,
wherein said [[2-[[-4-(3-hydroxyphenyl)-3,4-dimethylpiperidin-1-yl]methyl]-3-phenylpropanoyl]amino]acetic acid is in hydrate form.

3. A method according to claim 2,
wherein said [[2-[[-4-(3-hydroxyphenyl)-3,4-dimethylpiperidin-1-yl]methyl]-3-phenylpropanoyl]amino]acetic acid is [[2-[[-4-(3-hydroxyphenyl)-3,4-dimethylpiperidin-1-yl]methyl]-3-phenylpropanoyl]amino]acetic acid dihydrate.

4. A method according to claim 3,
wherein said compound is a substantially pure stereoisomer.

5. A method according to claim 4,
wherein said [[2-[[-4-(3-hydroxyphenyl)-3,4-dimethylpiperidin-1-yl]methyl]-3-phenylpropanoyl]amino]acetic acid is [[(2S)-2-[[(3R,4R)-4-(3-hydroxyphenyl)-3,4-dimethylpiperidin-1-yl]methyl]-3-phenylpropanoyl]amino]acetic acid dihydrate.

6. A method according to claim 1,
wherein said 4-aryl-piperidine derivative or stereoisomer, prodrug, pharmaceutically acceptable salt, hydrate, solvate, acid salt hydrate, N-oxide or isomorphic crystalline form thereof is administered at a level of at least about 0.75 mg/day.

7. A method according to claim 6,
wherein said 4-aryl-piperidine derivative or stereoisomer, prodrug, pharmaceutically acceptable salt, hydrate, solvate, acid salt hydrate, N-oxide or isomorphic crystalline form thereof is administered at a level of at least about 1 mg/day.

8. A method according to claim 7,
wherein said 4-aryl-piperidine derivative or stereoisomer, prodrug, pharmaceutically acceptable salt, hydrate, solvate, acid salt hydrate, N-oxide or isomorphic crystalline form thereof is administered at a level of at least about 2 mg/day.

9. A method according to claim 8,
wherein said 4-aryl-piperidine derivative or stereoisomer, prodrug, pharmaceutically acceptable salt, hydrate, solvate, acid salt hydrate, N-oxide or isomorphic crystalline form thereof is administered at a level of at least about 3 mg/day.

10. A method according to claim 1,
wherein said 4-aryl-piperidine derivative or stereoisomer, prodrug, pharmaceutically acceptable salt, hydrate, solvate, acid salt hydrate, N-oxide or isomorphic crystalline form thereof is administered at a level of less than about 15 mg/day.

11. A method according to claim 10,
wherein said 4-aryl-piperidine derivative or stereoisomer, prodrug, pharmaceutically acceptable salt, hydrate, solvate, acid salt hydrate, N-oxide or isomorphic crystalline form thereof is administered at a level of less than about 12 mg/day.

12. A method according to claim 11,
wherein said 4-aryl-piperidine derivative or stereoisomer, prodrug, pharmaceutically acceptable salt, hydrate, solvate, acid salt hydrate, N-oxide or isomorphic crystalline form thereof is administered at a level of less than about 9 mg/day.

13. A method according to claim 12,
wherein said 4-aryl-piperidine derivative or stereoisomer, prodrug, pharmaceutically acceptable salt, hydrate, solvate, acid salt hydrate, N-oxide or isomorphic crystalline form thereof is administered at a level of less than about 6 mg/day.

14. A method according to claim 1,
wherein said 4-aryl-piperidine derivative or stereoisomer, prodrug, pharmaceutically acceptable salt, hydrate, solvate, acid salt hydrate, N-oxide or isomorphic crystalline form thereof is administered for at least about 1 day.

15. A method according to claim 14,
wherein said 4-aryl-piperidine derivative or stereoisomer, prodrug, pharmaceutically acceptable salt, hydrate, solvate, acid salt hydrate, N-oxide or isomorphic crystalline form thereof is administered for at least about 2 days.

16. A method according to claim 15,
wherein said 4-aryl-piperidine derivative or stereoisomer, prodrug, pharmaceutically acceptable salt, hydrate, solvate, acid salt hydrate, N-oxide or isomorphic crystalline form thereof is administered for at least about 3 days.

17. A method according to claim 16,
wherein said 4-aryl-piperidine derivative or stereoisomer, prodrug, pharmaceutically acceptable salt, hydrate, solvate, acid salt hydrate, N-oxide or isomorphic crystalline form thereof is administered for at least about 5 days.

18. A method according to claim 17,
wherein said 4-aryl-piperidine derivative or stereoisomer, prodrug, pharmaceutically acceptable salt, hydrate, solvate, acid salt hydrate, N-oxide or isomorphic crystalline form thereof is administered for at least about 7 days.

19. A method according to claim 1,
wherein said gastrointestinal dysfunction is chronic constipation.

20. A method according to claim 19,
wherein said chronic constipation is associated with irritable bowel syndrome.

21. A method according to claim 1,
wherein said gastrointestinal dysfunction is slow colonic transit.

22. A method according to claim 21,
wherein said administration does not substantially affect the oral-cecal transit time.

23. A method according to claim 1,
wherein said gastrointestinal dysfunction is low stool frequency.

24. A method according to claim 23,
wherein said gastrointestinal dysfunction is poor stool consistency.

25. A method according to claim 1,
wherein said 4-aryl-piperidine derivative or stereoisomer, prodrug, pharmaceutically acceptable salt, hydrate, solvate, acid salt hydrate, N-oxide or isomorphic crystalline form thereof is administered at a level of about 0.5 mg/day to about 10 mg/day; and
wherein said gastrointestinal dysfunction is chronic constipation.

26. A method according to claim 25,
wherein said chronic constipation is associated with irritable bowel syndrome.

27. A method of treating or preventing non-opioid induced gastrointestinal dysfunction, comprising the step of:
administering to a patient in need thereof about 0.5 mg/day to about 18 mg/day of at least one 4-aryl-piperidine derivative or a stereoisomer, a prodrug, a pharmaceutically acceptable salt, a hydrate, a solvate, an acid salt hydrate, an N-oxide or an isomorphic crystalline form thereof;
wherein said patient is not receiving chronic or periodic exogenous opioids;
wherein said gastrointestinal dysfunction is chronic constipation, slow colonic transit, low stool frequency, poor stool consistency, or combinations thereof; and
wherein said 4-aryl-piperidine derivative is a compound of formula (IA): 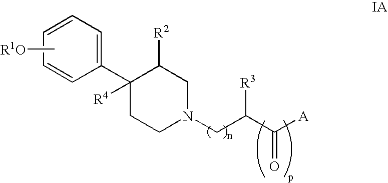
wherein:
R¹ is hydrogen or alkyl;
R² is hydrogen, alkyl or alkenyl;
R³ is hydrogen, alkyl, alkenyl, aryl, cycloalkyl, cycloalkenyl, cycloalkyl-substituted alkyl, cycloalkenyl-substituted alkyl or aralkyl;
R⁴ is hydrogen, alkyl or alkenyl;
A is OR⁵ or NR⁶R⁷;
R⁵ is hydrogen, alkyl, alkenyl, cycloalkyl, cycloalkenyl, cycloalkyl-substituted alkyl, cycloalkenyl-substituted alkyl, or aralkyl;
R⁶ is hydrogen or alkyl;
R⁷ is hydrogen, alkyl, alkenyl, cycloalkyl, aryl, cycloalkyl-substituted alkyl, cycloalkenyl, cycloalkenyl-substituted alkyl, aralkyl, B, or alkylene substituted B or, together with the nitrogen atom to which they are attached, R⁶ and R⁷ form a heterocyclic ring;
B is 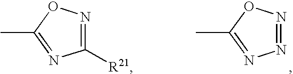
C(═O)W or NR⁸R⁹;
R⁸ is hydrogen or alkyl;
R⁹ is hydrogen, alkyl, alkenyl, cycloalkyl-substituted alkyl, cycloalkyl, cycloalkenyl, cycloalkenyl-substituted alkyl, aryl or aralkyl or, together with the nitrogen atom to which they are attached, R⁸ and R⁹ form a heterocyclic ring;
W is OR¹⁰, NR¹¹R¹², or OE;
R¹⁰ is hydrogen, alkyl, alkenyl, cycloalkyl, cycloalkenyl, cycloalkyl-substituted alkyl, cycloalkenyl-substituted alkyl, or aralkyl;
R¹¹ is hydrogen or alkyl;
R¹² is hydrogen, alkyl, alkenyl, aryl, cycloalkyl, cycloalkenyl, cycloalkyl-substituted alkyl, cycloalkenyl-substituted alkyl, aralkyl or alkylene substituted C(═O)Y or, together with the nitrogen atom to which they are attached, R¹¹ and R¹² form a heterocyclic ring;
E is 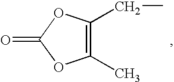
alkylene substituted (C═O)D, or —R¹³OC(═O)R¹⁴;
R¹³ is alkyl substituted alkylene;
R¹⁴ is alkyl;
D is OR¹⁵ or NR¹⁶R¹⁷;
R¹⁵ is hydrogen, alkyl, alkenyl, cycloalkyl, cycloalkenyl, cycloalkyl-substituted alkyl, cycloalkenyl-substituted alkyl, or aralkyl;
R¹⁶ is hydrogen, alkyl, alkenyl, aryl, aralkyl, cycloalkyl, cycloalkenyl, cycloalkyl-substituted alkyl or cycloalkenyl-substituted alkyl;
R¹⁷ is hydrogen or alkyl or, together with the nitrogen atom to which they are attached, R¹⁶ and R¹⁷ form a heterocyclic ring;
Y is OR¹⁸ or NR¹⁹R²;
R¹⁸ is hydrogen, alkyl, alkenyl, cycloalkyl, cycloalkenyl, cycloalkyl-substituted alkyl, cycloalkenyl-substituted alkyl, or aralkyl;
R¹⁹ is hydrogen or alkyl;
R²⁰ is hydrogen, alkyl, alkenyl, aryl, cycloalkyl, cycloalkenyl, cycloalkyl-substituted alkyl, cycloalkenyl-substituted alkyl, or aralkyl or, together with the nitrogen atom to which they are attached, R¹⁹ and R²⁰ form a heterocyclic ring;
R²¹ is hydrogen or alkyl;
n is 0 to 4;
p is 0 or 1; and
provided that R¹⁰ is not hydrogen, when R¹ is hydrogen, R² is methyl, R³ is cycloalkyl-substituted alkyl, and R⁴ is methyl; and
provided that R¹⁰ is not alkyl, when R¹ is hydrogen, R² is methyl, R³ is aralkyl, and R⁴ is methyl.

28. A method according to claim 27,
wherein the compound of formula (IA) is a trans 3,4-isomer.

29. A method according to claim 27,
wherein:
R¹ is hydrogen;
R² is alkyl;
n is 1 or 2;
R³ is benzyl, phenyl, cyclohexyl, or cyclohexylmethyl; and
R⁴ is alkyl.

30. A method according to claim 27,
wherein:
A is OR⁵; and
R⁵ is hydrogen or alkyl.

31. A method according to claim 27,
wherein:
A is NR⁶R⁷;
R⁶ is hydrogen;
R⁷ is alkylene substituted B; and
B is C(O)W.

32. A method according to claim 27,
wherein:
R⁷ is (CH₂)_{q}—B;
q is about 1 to about 3;
W is OR¹⁰; and
R¹⁰ is hydrogen, alkyl, phenyl-substituted alkyl, cycloalkyl or cycloalkyl-substituted alkyl.

33. A method according to claim 27,
wherein:
W is NR¹¹R¹²
R¹¹ is hydrogen or alkyl; and
R¹² is hydrogen, alkyl or alkylene substituted C(═O)Y.

34. A method according to claim 27,
wherein:
R¹² is (CH₂)ₘC(O)Y;
m is 1 to 3;
Y is OR¹⁸ or NR¹⁹R²⁰; and
R¹⁸, R¹⁹ and R²⁰ are independently hydrogen or alkyl.

35. A method according to claim 27,
wherein:
W is OE;
E is CH₂C(═O)D;
D is OR⁵ or NR¹⁶R¹⁷;
R¹⁵ is hydrogen or alkyl;
R¹⁶ is methyl or benzyl; and
R¹⁷ is hydrogen.

36. A method according to claim 27,
wherein:
W is OE;
E is R¹³OC(═O)R¹⁴;
R¹³ is —CH(CH₃)— or —CH(CH₂CH₃)—; and
R¹⁴is alkyl.

37. A method according to claim 27,
wherein p is 1.

38. A method according to claim 27,
wherein the configuration at positions 3 and 4 of the piperidine ring is each R.

39. A method according to claim 27,
wherein said compound is selected from the group consisting of:
Q-CH₂CH(CH₂(C₆H₅))C(O)OH,
Q-CH₂CH₂CH(C₆H₅)C(O)NHCH₂C(O)OCH₂CH₃,
Q-CH₂CH₂CH(C₆H₅)C(O)NHCH₂C(O)OH,
Q-CH₂CH₂CH(C₆H₅)C(O)NHCH₂C(O)NHCH₃,
Q-CH₂CH₂CH(C₆H₅)C(O)NHCH₂C(O)NHCH₂CH₃,
G-NH(CH₂)₂C(O)NH₂,
G-NH(CH₂)₂C(O)NHCH₃,
G-NHCH₂C(O)NH₂,
G-NHCH₂C(O)NHCH₃,
G-NHCH₂C(O)NHCH₂CH₃,
G-NH(CH₂)₃C(O)OCH₂CH₃,
G-NH(CH₂)₃C(O)NHCH₃,
G-NH(CH₂)₂C(O)OH,
G-NH(CH₂)₃C(O)OH,
Q-CH₂CH(CH₂(C₆H₁₁))C(O)NH(CH₂)₂C(O)OH,
Q-CH₂CH(CH₂(C₆H₁₁))C(O)NH(CH₂)₂C(O)NH₂,
Z-NHCH₂C(O)OH,
Z-NHCH₂C(O)NH₂,
Z-NHCH₂C(O)N(CH₃)₂,
Z-NHCH₂C(O)NHCH(CH₃)₂,
Z-NH(CH₂)₂C(O)OCH₂(C₆H₅),
Z-NH(CH₂)₂C(O)NHCH₂CH₃,
Z-NH(CH₂)₃C(O)NHCH₃,
Z-NHCH₂C(O)NHCH₂C(O)OH,
Z-NHCH₂C(O)OCH₂C(O)OCH₃,
Z-NHCH₂C(O)OCH₂C(O)NHCH₃,
Z-NHCH₂C(O)O-(4-methoxycyclohexyl),
Z-NHCH₂C(O)OCH₂C(O)NHCH₂(C₆H₅) and
Z-NHCH₂C(O)OCH(CH₃)OC(O)CH₃;
wherein:

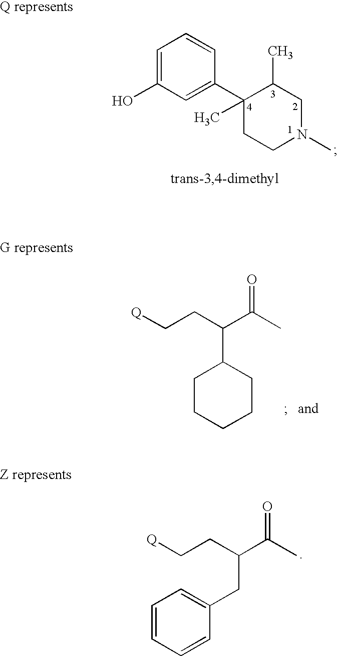

40. A method according to claim 39,
wherein said compound is selected from the group consisting of:
(+)-Z-NHCH₂C(O)OH,
(−)-Z-NHCH₂C(O)OH,
(3R,4R)-Z-NHCH₂C(O)NHCH₂(C₆H₅) and
(3R,4R)-G-NH(CH₂)₃C(O)OH.

41. A method according to claim 40,
wherein said compound is selected from the group consisting of:
(+)-Z-NHCH₂C(O)OH, and
(−)-Z-NHCH₂C(O)OH.

42. A method according to claim 41,
wherein said compound is selected from the group consisting of:
(+)-Z-NHCH₂C(O)OH.

43. A method according to claim 40,
wherein said compound is Q-CH₂CH(CH₂(C₆H₅))C(O)OH.

44. A method according to claim 43,
wherein said compound is (3R,4R,S)-Q-CH₂CH(CH₂(C₆H₅))C(O)OH.

45. A method according to claim 27,
wherein said compound is a substantially pure stereoisomer.

46. A method according to claim 27,
wherein said 4-aryl-piperidine derivative or stereoisomer, prodrug, pharmaceutically acceptable salt, hydrate, solvate, acid salt hydrate, N-oxide or isomorphic crystalline form thereof is administered at a level of at least about 0.75 mg/day.

47. A method according to claim 46,
wherein said 4-aryl-piperidine derivative or stereoisomer, prodrug, pharmaceutically acceptable salt, hydrate, solvate, acid salt hydrate, N-oxide or isomorphic crystalline form thereof is administered at a level of at least about 1 mg/day.

48. A method according to claim 47,
wherein said 4-aryl-piperidine derivative or stereoisomer, prodrug, pharmaceutically acceptable salt, hydrate, solvate, acid salt hydrate, N-oxide or isomorphic crystalline form thereof is administered at a level of at least about 2 mg/day.

49. A method according to claim 48,
wherein said 4-aryl-piperidine derivative or stereoisomer, prodrug, pharmaceutically acceptable salt, hydrate, solvate, acid salt hydrate, N-oxide or isomorphic crystalline form thereof is administered at a level of at least about 3 mg/day.

50. A method according to claim 27,
wherein said 4-aryl-piperidine derivative or stereoisomer, prodrug, pharmaceutically acceptable salt, hydrate, solvate, acid salt hydrate, N-oxide or isomorphic crystalline form thereof is administered at a level of less than about 15 mg/day.

51. A method according to claim 50, wherein said 4-aryl-piperidine derivative or stereoisomer, prodrug, pharmaceutically acceptable salt, hydrate, solvate, acid salt hydrate, N-oxide or isomorphic crystalline form thereof is administered at a level of less than about 12 mg/day.

52. A method according to claim 51,
wherein said 4-aryl-piperidine derivative or stereoisomer, prodrug, pharmaceutically acceptable salt, hydrate, solvate, acid salt hydrate, N-oxide or isomorphic crystalline form thereof is administered at a level of less than about 9 mg/day.

53. A method according to claim 52,
wherein said 4-aryl-piperidine derivative or stereoisomer, prodrug, pharmaceutically acceptable salt, hydrate, solvate, acid salt hydrate, N-oxide or isomorphic crystalline form thereof is administered at a level of less than about 6 mg/day.

54. A method according to claim 27,
wherein said 4-aryl-piperidine derivative or stereoisomer, prodrug, pharmaceutically acceptable salt, hydrate, solvate, acid salt hydrate, N-oxide or isomorphic crystalline form thereof is administered for at least about 1 day.

55. A method according to claim 54,
wherein said 4-aryl-piperidine derivative or stereoisomer, prodrug, pharmaceutically acceptable salt, hydrate, solvate, acid salt hydrate, N-oxide or isomorphic crystalline form thereof is administered for at least about 2 days.

56. A method according to claim 55,
wherein said 4-aryl-piperidine derivative or stereoisomer, prodrug, pharmaceutically acceptable salt, hydrate, solvate, acid salt hydrate, N-oxide or isomorphic crystalline form thereof is administered for at least about 3 days.

57. A method according to claim 56,
wherein said 4-aryl-piperidine derivative or stereoisomer, prodrug, pharmaceutically acceptable salt, hydrate, solvate, acid salt hydrate, N-oxide or isomorphic crystalline form thereof is administered for at least about 5 days.

58. A method according to claim 57,
wherein said 4-aryl-piperidine derivative or stereoisomer, prodrug, pharmaceutically acceptable salt, hydrate, solvate, acid salt hydrate, N-oxide or isomorphic crystalline form thereof is administered for at least about 7 days.

59. A method according to claim 27,
wherein said gastrointestinal dysfunction is chronic constipation.

60. A method according to claim 59,
wherein said chronic constipation is associated with irritable bowel syndrome.

61. A method according to claim 27,
wherein said gastrointestinal dysfunction is slow colonic transit.

62. A method according to claim 61,
wherein said administration does not substantially affect the oral-cecal transit time.

63. A method according to claim 27,
wherein said gastrointestinal dysfunction is low stool frequency.

64. A method according to claim 27,
wherein said gastrointestinal dysfunction is poor stool consistency.

65. A method according to claim 27,
wherein said 4-aryl-piperidine derivative or stereoisomer, prodrug, pharmaceutically acceptable salt, hydrate, solvate, acid salt hydrate, N-oxide or isomorphic crystalline form thereof is administered at a level of about 0.5 mg/day to about 10 mg/day; and
wherein said gastrointestinal dysfunction is chronic constipation.

66. A method according to claim 65,
wherein said chronic constipation is associated with irritable bowel syndrome.
